SEQUENCE LISTING (29 sequences)

SEQ ID NO: 1 - DNA, 20 bp, Artificial Sequence
  Description of Artificial Sequence Synthetic primer
tgtgcacaggagccaagagt

SEQ ID NO: 2 - DNA, 21 bp, Artificial Sequence
  Description of Artificial Sequence Synthetic primer
attttcttgctgccagtctgg

SEQ ID NO: 3 - DNA, 18 bp, Artificial Sequence
  Description of Artificial Sequence Synthetic primer
cctttcccatggatgaag

SEQ ID NO: 4 - DNA, 18 bp, Artificial Sequence
  Description of Artificial Sequence Synthetic primer
cgtccgcttgttctcctc

SEQ ID NO: 5 - DNA, 24 bp, Artificial Sequence
  Description of Artificial Sequence Synthetic primer
aagcatttactttgtggctggatt

SEQ ID NO: 6 - DNA, 25 bp, Artificial Sequence
  Description of Artificial Sequence Synthetic primer
tgatctggatttctcctctgtgtct

SEQ ID NO: 7 - DNA, 19 bp, Artificial Sequence
  Description of Artificial Sequence Synthetic primer
tgcagatgctggacgacag

SEQ ID NO: 8 - DNA, 23 bp, Artificial Sequence
  Description of Artificial Sequence Synthetic primer
gaactctgccaggatctgctcta

SEQ ID NO: 9 - DNA, 21 bp, Artificial Sequence
  Description of Artificial Sequence Synthetic primer
tgaacaccagagagtccagca

SEQ ID NO: 10 - DNA, 19 bp, Artificial Sequence
  Description of Artificial Sequence Synthetic primer
gcttggctggtggcttctt

SEQ ID NO: 11 - DNA, 20 bp, Artificial Sequence
  Description of Artificial Sequence Synthetic primer
ccccagactccgtcagtttc

SEQ ID NO: 12 - DNA, 19 bp, Artificial Sequence
  Description of Artificial Sequence Synthetic primer
tccgtctggttgggttcag

SEQ ID NO: 13 - DNA, 21 bp, Artificial Sequence
  Description of Artificial Sequence Synthetic primer
aagaggccatcaagcagatca

SEQ ID NO: 14 - DNA, 17 bp, Artificial Sequence
  Description of Artificial Sequence Synthetic primer
caggaggcgcatccaca

SEQ ID NO: 15 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 16 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 17 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 18 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 19 - protein, 723 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 20 - protein, 728 aa, Homo sapiens; identical to SEQ ID NO: 18

SEQ ID NO: 21 - protein, 87 aa, Homo sapiens
MKIILWLCVFGLFLATLFPISWQMPVESGLSSEDSASSESFASKIKRHGEGTFTSDLSKQMEEEAVRLFIEWLKNGGPSSGAPPPSG

SEQ ID NO: 22 - protein, 87 aa, Homo sapiens; identical to SEQ ID NO: 21

SEQ ID NO: 23 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 24 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 25 - protein, 232 aa, Homo sapiens; identical to SEQ ID NO: 24

SEQ ID NO: 26 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 27 - protein, 180 aa, Homo sapiens; identical to SEQ ID NO: 26

SEQ ID NO: 28 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 29 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

(19) United States
(12) Patent Application Publication
(10) Pub. No.: US 2017/0240864 A1
(43) Pub. Date: Aug. 24, 2017

(54) METHODS TO GENERATE PANCREATIC BETA CELLS FROM SKIN CELLS

(71) Applicant: UNIVERSITY OF IOWA RESEARCH FOUNDATION, Iowa City, IA (US)

(72) Inventors: Nicholas Zavazava, Coralville, IA (US); Gohar Shahwar Manzar, Rochester, MN (US); Sudhanshu Premchand Raikwar, Coralville, IA (US); Eun-Mi Kim, Iowa City, IA (US)

(73) Assignee: UNIVERSITY OF IOWA RESEARCH FOUNDATION, Iowa City, IA (US)

(21) Appl. No.: 15/418,498

(22) Filed: Jan. 27, 2017

Related U.S. Application Data
(60) Provisional application No. 62/287,768, filed on Jan. 27, 2016.

(51) Int. Cl. C12N 5/071

(52) U.S. Cl. CPC C12N 5/0676; C12N 2501/105; C12N 2506/09; C12N 2501/395; C12N 2501/91; C12N 2501/415; C12N 2501/117; C12N 2501/40; C12N 2501/12; C12N 2501/15; C12N 2501/33; C12N 2501/998; C12N 2500/38

(57) ABSTRACT

In certain embodiments, the present invention provides methods to prepare insulin secreting cells from a skin sample from a mammal.

### RELATED APPLICATION

This application claims priority to U.S. Provisional Patent Application No. 62/287,768, filed Jan. 27, 2016, the entirety of which is incorporated herein by reference.

### GOVERNMENT SUPPORT

The invention was made with government support under #1IO1BX001125-01A1 awarded by the Department of Veterans Affairs, and NHLBI #R01 HLO73015 and NIDDK #P30 DK054759 awarded by the National Institutes of Health. The Government has certain rights in the invention.

### BACKGROUND

Type 1 diabetes (T1D) is a chronic, debilitating autoimmune disease targeting pancreatic β-cells diminishing the β-cell mass resulting in insufficient insulin production (Leroux et al., 2014). Although the exact etiology of T1D remains unknown, it is prevalent among young children and remains without a practical cure, despite being one of the most common endocrine disorders. Successful transplantation of pancreatic islets or the pancreas can reduce some complications of T1D. However, organ shortage severely limits this approach.

Pluripotent stem cells (PSCs) potentially address many of these shortcomings since their availability is unlimited and they are considered to be immune privileged (Drukker et al, 2003; Kim et al., 2013; Robinton et al, 2012). Both induced pluripotent stem (iPS) and ES cells are capable of forming any cell type when exposed to appropriate cues. However, unlike ES cells that require the controversial destruction of embryos, iPS cells are derived from pre-existing somatic cells, which allows one to design patient-tailored therapies (Robinton et al., 2012). These characteristics provide the unique opportunity to engineer autologous insulin producing cells (IPCs) that can be used to replace β-cells destroyed in T1D (Monzar et al., 2014). Moreover, these cells have enormous value in drug screening as well as in modeling the pathogenesis of T1D, eliminating the need for animal studies that poorly correlate with studies in humans.

The process of differentiating ES or iPS cells into pancreatic endocrine cells in vitro mimics the developmental stages observed during embryogenesis (Spence et al., 2007). Overall, it has proven challenging to generate mature and functional IPCs from pancreatic stem cells (PSCs) that possess hallmark features of adult pancreatic β-cells, such as glucose responsiveness and rapid correction of hyperglycemia (D'Amour et al., 2006; Kroon et al., 2008; Rezania et al., 2012; Xie et al., 2013; Zhang et al., 2009). More recently, two reports devised strategies to differentiate human ES and iPS cells into glucose-responsive IPCs that strongly resembled adult pancreatic β-cells (Pagliuca et al., 2014; Rezania et al., 2014). Unfortunately, the rate of glucose correction after transplantation of the cells into diabetic mice was still rather slow.

### SUMMARY

The hurdles in the generation of IPCs from iPS cells may be overcome by replacing 2D culture systems with a 3D differentiation culture system, selecting sets of factors applied to cells in stepwise fashion, and optionally treating cells at certain points in differentiation with one or more demethylation agents. The number of IPCs derived from 3D culture systems was found to be superior to what had been described in the literature. The reason for this improvement is that, during embryogenesis, the developing cells are arranged in 3D clusters, which support cell-cell signaling. Here, 3D cultures were established using matrigel to exploit scaffold-embedded signaling cues. By combining a 3D bio-scaffold-based culture platform with signaling cues, the efficiency of generating glucose-responsive IPCs was improved.

iPS cells derived from T1D patients have been shown to have a lower efficiency in generating pancreatic progenitor cells expressing Pdx1. If this is common to all T1D cell lines, autologous iPS cell therapy for T1D will be a challenge. However, as described herein below a protocol was established that significantly improves the differentiation of T1D iPS cells into IPCs.

In one embodiment, a method is provided by which induced pluripotent stem (iPS) cells are converted into pancreatic beta cells. In one embodiment, a skin biopsy is obtained from a diabetic patient and skin cells are isolated. These skin cells are converted into induced pluripotent stem (iPS) cells. Then the iPS cells are subjected to conditions so that the iPS cell become like pancreatic beta cells that secrete insulin, the hormone that regulates blood sugar. These pancreatic cells regulated blood sugar levels in diabetic mice. This approach may eliminate the need for cadaveric tissues. Moreover, an outstanding advantage of the method is that there would be no wait time since the skin cells of the patient are always available. In addition, since the cells are from the same patient, the need for immunosuppression is eliminated. Further, screening of these cells with individual drugs may help to target treatment.

This invention thus provides for the inhibition or treatment of diabetes, allowing for individualized treatments of diabetes with the patient's own cells. The cells generated are useful for drug screening and pretesting of drugs. Also, since there is a chronic shortage of organs because number of patients far outweighs the number of available donors, the present method allows for an alternative to organ donation.

In one embodiment, a method is provided to prepare insulin secreting cells from a skin sample from a mammal. The method includes: providing a sample of skin cells from a mammal; subjecting the skin cells to conditions that convert the skin cells to induced pluripotent stem cells; and treating the induced pluripotent stem cells to a plurality of agents that are sequentially applied and result in stepwise differentiation of the induced pluripotent stem cells to insulin secreting cells. In one embodiment, cells at one or more stages in differentiation are treated with a demethylation agent. In one embodiment, cells at one or more stages in differentiation are cultured in or on a gelatinous protein mixture. In one embodiment, the cells are human cells. In one embodiment, the mammal is a human that has type 1 diabetes. In one embodiment, the steps to induce differentiation include differentiating the induced pluripotent stem cells to definitive endodermal cells, differentiating the definitive endodermal cells to posterior foregut cells, differentiating the posterior foregut cells to pancreatic endodermal or progenitor cells, differentiating the pancreatic endodermal or progenitor cells to endocrine precursors, and differentiating the endocrine precursor cells to insulin producing cells. In one embodiment, the cells are treated with at least one of keratinocyte growth factor (KGF), L-ascorbic acid, or Y27632, or any combination thereof. In one embodiment, the cells are treated with at least one of a KGFR agonist, L-ascorbic acid or an analog or Rho-associated kinase inhibitor, or any combination thereof. In one embodiment, the cells are treated with at least one of SANT-1, retinoic acid, Noggin, B27, TPB, L-ascorbic acid, or keratinocyte growth factor, or any combination thereof. In one embodiment, the cells are treated with at least one of a Smo inhibitor and Sonic Hedgehog signaling pathway antagonist, retinoic acid, an inactivator of TGF-beta superfamily signaling proteins, B27, a PKC activator, L-ascorbic acid or an analog thereof, or a KGFR agonist, or any combination thereof. In one embodiment, the cells are treated with at least one of ALK5i, Noggin, B27 Supplement, glucagon like peptide-1 (GLP1), SANT1, retinoic acid, DAPT or heparin, or any combination thereof, followed by treatment with ALK5i, Noggin, B27 Supplement, GLP1, DAPT, heparin, or T3, or any combination thereof. In one embodiment, the cells are treated with at least one of an inhibitor of TGF-beta RI kinase, an inactivator of TGF-beta superfamily signaling proteins, B27 Supplement, GLP1, a Smo inhibitor and antagonist of Sonic Hedgehog signaling, retinoic acid or an analog thereof, an inhibitor of gamma-secretase complex, or heparin or an analog thereof, or any combination thereof, followed by treatment with an inhibitor of TGF-beta RI kinase, an inactivator of TGF-beta superfamily signaling proteins, B27 Supplement, GLP1, an inhibitor of gamma-secretase complex, or heparin, or any combination thereof, followed by treatment with an inhibitor of TGF-beta RI kinase, heparin, or T3, or any combination thereof. In one embodiment, the cells are treated with at least one of nicotinamide, IGF-1, GLP-1, ALK5i, T3, or heparin, or any combination thereof. In one embodiment, the cells are treated with at least one of nicotinamide or an analog thereof, IGF-1, GLP-1, an inhibitor of TGF-beta RI kinase, T3 or an analog thereof, or heparin, or any combination thereof. In one embodiment, the cells that are produced are glucose responsive and, when transplanted into a diabetic mammal, allows for correction of hyperglycemia, normalization of blood glucose levels, e.g., within 2 to 4 weeks after transplant, and normalization of insulin expression, e.g., relative to a corresponding non-diabetic mammal.

In one embodiment, the steps to induce differentiation include treating induced pluripotent stem cells with at least one of Activin A or an analog thereof or Wnt3a or an analog thereof, or a combination thereof, thereby providing definitive endodermal cells; treating the definitive endodermal cells with at least Activin A or an analog thereof, and introducing the treated definitive endodermal cells to a gelatin coated substrate and are treated at least one of keratinocyte growth factor or an analog thereof, Noggin or an analog thereof, or B27 having insulin but lacking vitamin A, or any combination thereof, thereby providing pancreatic endoderm cells; and treating the pancreatic endoderm cells with at least one of HGF or an analog thereof, exendin-4 or an analog thereof, or nicotinamide or an analog thereof, or any combination thereof. In one embodiment, the steps to induce differentiation include treating induced pluripotent stem cells with at least one of a TGF-beta family member or a Wnt family member, or both, thereby providing definitive endodermal cells; treating the definitive endodermal cells with at least a TGF-beat family member and introducing the treated definitive endodermal cells to a gelatin coated substrate and are treated at least one of a keratinocyte growth factor receptor agonist, an inactivator of TGF-beta superfamily member signaling proteins, or B27 having insulin but lacking vitamin A, or any combination thereof, thereby providing pancreatic endoderm cells; and treating the pancreatic endoderm cells with at least one of HGF or an analog thereof, exendin-4 or an analog thereof, or nicotinamide or an analog thereof, or any combination thereof.

### BRIEF DESCRIPTION OF FIGURES

FIGS. 1A-1C. Generation of IPCs. FIG. 1A) Stepwise differentiation of human iPS cells leads to the generation of definitive endodermal (DE) cells, pancreatic endodermal (PE) cells, pro-endocrine progenitors (S3), pre-IPCs (Pre), and finally insulin producing cells (IPCs).

FIG. 1B) At the endodermal stage, the cells were stained for CXCR4 and sorted using immunomagnetic beads. These cells were further used to differentiate IPCs. FIG. 1C) Precursor cells were stained for Pdx1 to determine whether they had adopted the pancreatic lineage. Almost all the cells were expressing Pdx1.

FIG. 2. Gene expression at various stages during the differentiation. Real-time quantitative PCR analysis at different differentiation stages revealed stage specific upregulation of genes involved in pancreatic beta cell development. In this diagram S1 represents DE cells, S2 represents PE cells, S3 represents pro-endocrine progenitors and Pre represents pre-IPCs. Human pancreatic islets and mouse βTC3 cells were used as positive controls while the T cell line NIH3T3 was used as negative control.

FIGS. 3A-3B. Characterization of IPCs. FIG. 3A) Real-time bioluminescence imaging and Dithizone staining were performed to confirm the generation of IPCs. The undifferentiated human iPS cells were transfected with a RIP-Luc expression vector. The cells at various stages of differentiation were subjected to real-time bioluminescence imaging. The undifferentiated cells as well as DE cells (S1) failed to generate detectable bioluminescence signal. However, very faint bioluminescence signal was detected in the PE cells (S2). The bioluminescence signal intensity progressively increased in the pro-endocrine progenitors (S3) and maximum bioluminescence signal intensity was observed in the human iPS cell-derived IPCs. FIG. 3B) The human iPS cell-derived IPCs were stained with dithizone stain. The IPCs strongly stained positive.

FIGS. 4A-4D Imaging of IPCs using Transmission Electron Microscopy. FIG. 4A) Human iPS cell derived-IPCs were positive for C-peptide as well as Marf A. FIG. 4B) Human iPS cell-derived IPCs as well as human pancreatic islets were analyzed by transmission electron microscopy to identify insulin secretory granules. In human iPS cell-derived IPCs a few (*50-70) insulin granules with and without characteristic halo around them were detected. In contrast, the human pancreatic islets had >100 insulin secretory granules with a characteristic halo around them (FIG. 4C). FIG. 4D) The number of granules in both human islets and the IPCs show that IPCs display fewer granules than those counted in the islets.

FIGS. 5A-5C. Immunostaining of IPCs. The human iPS cells undergoing differentiation were subjected to immunostaining at various stages. The differentiation led to generation of DE cells which were positive for Sox17 and Foxa2 (FIG. 5A). The PE cells were positive for Pdx1 and Nkx2.2 (FIG. 5B). The islet-like clusters were positive for C-peptide as well as glucagon (FIG. 5C).

FIGS. 6A-6B. IPCs respond to high glucose levels as detected by the mitochondria stress test. Human iPS cell-derived IPCs were tested for oxygen consumption using the mitochondria stress test. In high glucose (FIG. 6A), oxygen consumption significantly went up and could be blocked by Nifedipine. As expected, in low glucose (FIG. 6B) oxygen consumption could not be increased by IBMX (3-isobutyl-1-methylxanthine).

FIGS. 7A-7C. IPCs correct hyperglycemia in diabetic mice. FIG. 7A) Eight weeks old Rag2^{-/-}γc^{-/-} mice were made diabetic following streptozotocin treatment. The pre-transplant blood levels were >400 mg/dl. Approximately 5×10⁶ human iPS cell-derived IPCs were transplanted under the kidney capsule of each mouse. The blood glucose levels were monitored for >100 days. Blood glucose levels with peak and trough kinetics were observed throughout the duration of the study. The normalization of blood glucose levels was observed in 3 out of 5 mice. The remaining 2 were borderline diabetic. FIG. 7B) To study whether the transplanted mice could control high sugar levels, we performed the glucose tolerance test. Mice that had become normoglycemic were subjected to intraperitoneal glucose tolerance test. The normal healthy control mice displayed a faster blood glucose clearance while the mice transplanted with human iPS cell-derived IPCs had a slightly impaired glucose tolerance. FIG. 7C) To rule out that the pancreata of streptozotocin-treated mice did not regenerate, we sacrificed the transplanted mice and histologically examined the pancreas. Streptozotocin-treated mice that had received HPCs did not have any pancreatic islets left.

FIGS. 8A-8C. IPCs form organoids in vivo under the kidney capsule. FIG. 8A) The Rag2^{-/-}γc^{-/-} mice transplanted with human iPS cell-derived IPCs were subjected to MRI to monitor the fate of the transplanted IPCs in real-time. MRI revealed that the transplanted IPCs were present as a white mass on the surface of the kidney they were transplanted into. Kidneys were imaged both in the axial and coronal projections. FIG. 8B) To further study the tissue on the kidneys, the mice were sacrificed. The transplanted IPCs were observed as a white vascularized organoid with triangular shape. There was no evidence of any teratoma formation or abnormal growth. The IPC transplanted kidney showed signs of tissue adhesion at the site of IPC injection (dotted line). FIG. 8C) To examine the organoids further, histological sections were stained by H&E and studied under the microscope. The organoids appeared triangular in shape and separate from the kidneys. More interestingly they appeared to have developed neovascularization (insert, arrow heads).

FIGS. 9A-9F. Histology and Immunostaining of the transplanted IPCs. To confirm that the organoids were pancreatic in nature, histological sections were stained for C-peptide (FIG. 9A), insulin (FIG. 9B, FIG. 9E), isotype control (FIG. 9C) and glucagon (FIG. 9D). The overlay (FIG. 9F) shows that the cells are positive for both hormones suggesting that they are bihormonal.

FIGS. 10A-10D. Solid spheroids but not hollow cysts found predominantly in T1D differentiating cultures express insulin. FIG. 10A) This differentiation schema shows in a clockwise manner the five stages of differentiation that iPS cells undergo in order to become IPCs. FIG. 10B) T1D and ND iPS cells were differentiated in parallel under Stage 1 to generate CXCR4⁺ Sox17⁺ PDGFR-α⁻ DE cells. Over 90% of the resultant cells derived from both T1D and ND iPS cells co-expressed CXCR4 and Sox17, and almost all of these cells were PDGFR-α⁻, suggesting that they are endodermal but not mesendodermal. Undifferentiated iPS cells were used as negative controls for these stains. FIG. 10C) The parallel differentiation of T1D and ND iPS cells in 3D culture after Stage 1 results in distinct morphologies of hollow cyst-like (asterisk) and compact (arrowheads) IPC clusters towards the end of the differentiation. FIG. 10D) The hollow cysts prevalent in T1D IPC cultures, which collapse upon fixation, are insulin-negative (column 1). Insulin appears in compact cell clusters rarely found in T1D cultures (column 2) and more predominantly in the ND cultures (column 3). Controls for staining were iPS cells (not shown) and cadaveric human islets (column 4). Scale bar=50 μm.

FIG. 11. T1D iPS cells give rise to mostly hollow cyst-like clusters whereas ND iPS cells give rise to a mixture of hollow cysts and compact spheroids. Comparison of the ND and T1D cultures revealed significant disparities in the yield of these two cluster morphologies. Similar to fetal development of the pancreas, it was observed in both cases the presence of hollow vacuoles and tight spheroids. However, cells from the T1D patient consisted of significantly more hollow vacuoles than the cells from the ND patient, which had a nearly 50:50 mix of hollow cysts and compact spheroids (n=3 differentiations for ND cells and 8 for T1D cells). Data are represented as mean±SEM, **p<0.01.

FIG. 12. Rare large organoids in T1D IPC cultures express insulin. T1D iPS cells rarely yielded large, compact organoid-like structures measuring several millimeters in length. Immunostaining of these structures reveals strong expression of insulin. Scale bar=500 μm. A higher magnification of the staining reveals cytoplasmic staining of insulin in these rare structures. Scale bar=50 μm.

FIGS. 13A-13B. IPCs derived from T1D iPS cells poorly express Pdx1 and insulin. FIG. 13A) 50% of the ND IPCs are positive for insulin compared to only 15% of T1D IPCs. Negative controls were undifferentiated iPS cells and cadaveric human islets were used as positive controls. Remarkably, ND iPS cells yielded a population of insulin positive cells that was comparable to what was observed in cadaveric human islets (n=3). FIG. 13B) To investigate why the T1D cells poorly differentiated into IPCs, mRNA levels of various pancreatic genes were quantified in ND IPCs and T1D IPCs. Insulin expression in Stage 5 was accompanied by a striking decrease in Glucagon expression in the ND differentiating cultures. However, the T1D culture expressed significantly lower levels of Insulin and Glucagon compared to ND IPCs. Pdx1 expression in the T1D IPCs was significantly lower than in the ND IPCs (n=5). These data were generated by normalizing Ct values to an iPS cell line. The internal control used in this experiment was the TATA Binding Protein (TBP), which was used as a housekeeping gene. Data are represented as mean±SEM, *p<0.05, **p<0.01, ***p<0.001.

FIGS. 14A-14B. Selection of the optimal dose of 5-aza-DC to demethylate iPS cells while ensuring maximal cell viability. FIG. 14A) Every cell type has a unique tolerance to 5-aza-DC, which is toxic at high doses. In order to identify an optimal dose of 5-aza-DC that would maintain integrity of the cells while demethylating effectively, a dose screen experiment was conducted on T1D iPS cells (n=2), observing the quality of the colonies and degree of cell death over 4 days after 18 hours of treatment with various doses of 5-aza-DC. 30 nM of 5-aza-DC induced minimal toxicity (defined by thinning of colonies or loss of sharp colony edges), whereas drastically compromised integrity of the cultures was observed at 70 nM and 90 nM of 5-aza-DC. In order to ensure that the cultures will experience minimal toxicity, doses of 1 nM and 10 nM were subsequently used, mostly focusing on 10 nM. FIG. 14B) To confirm that those low doses would still demethylate cells, a dot blot for 5-methylcytosine was performed on gDNA isolated from untreated T1D iPS cells or iPS cells that were treated with 1 nM or 10 nM 5-aza-DC (n=3). Untreated iPS cells possessed significant 5-methylcytosine content (leftmost column), evidenced by the dark spot where the DNA was blotted. As can be observed by lightening of the spots at the 1 nM and 10 nM doses, 5-aza-DC appeared to effectively demethylate the cells (1 minute or 5 minute represents the exposure time for the blot).

FIG. 15. Demethylation on DO of differentiation arrests cells in a CXCR4+ PDGFRα+ Sox17− mesendodermal state. In order to identify the optimal time point at which demethylation should be initiated, two parallel differentiations of T1D iPS cells into DE cells were established. In one differentiation, the culture was exposed to 5-aza-DC on day 0 (which is the day that DE differentiation is initiated), whereas the other culture was demethylated at day 4 of differentiation (which is the last day of DE culture). At the end of the differentiations, the efficacy of DE cell differentiation was determined by studying the expression of CXCR4, PDGFR-α, and Sox17. Undifferentiated iPS cells (red plot) served as a negative control for all of these cell markers. Demethylation of the cells on day 0 of differentiation (green plot) resulted in cells that were CXCR4+ PDGFR-α+ Sox17−, which represents the immature mesendodermal state. In contrast, demethylation of the cells on day 4 (blue plot) generated >90% CXCR4+ Sox17+ PDGFR-α− cells, representing true DE cells. This result motivated implementation of demethylation at the end of Stage 1 (on day 4), after generating DE cells. Besides, after formation of the DE, the cells have a higher proliferative rate. Thus, temporal control of the demethylation treatment ensures enhanced differentiation outcomes.

FIGS. 16A-16E. Demethylation of T1D DE cells yields >90% Pdx1⁺ cells and >50% insulin-expressing cells while averting the generation of glucagon-expressing cells. FIG. 16A) Typically, T1D iPS cells give rise to a disorganized mix of cysts and spheroids (bottom half of leftmost column), with a dominant presence of hollow cysts. Treatment of DE cells with 5-aza-DC promotes the formation of compact clusters that uniquely resemble human islets in both size and morphology. Dithizone staining (n=4) reveals the strong red color of the compact clusters found in the 5-aza-DC treated cultures, which is reminiscent of islets. This is in contrast to what is observed in the untreated T1D IPC cultures, which stain brown in a manner similar to undifferentiated iPS cells. FIG. 16B) Demethylation of T1D DE cells resulted in >95% Pdx1⁺ cells at the end of Stage 4 (n=4). A significant proportion of the Pdx1⁺ cells at the end of Stage 4 co-expressed the pancreatic β-cell specific transcription factor Nkx6.1 (n=3). Undifferentiated iPS cells served as a negative control. FIG. 16C) Whereas untreated T1D iPS cells only yielded up to 1-13.7% insulin+ cells, in cultures treated with 5-Aza-DC, up to 56.7-59.4% of the cells were insulin-expressing (n=5). FIG. 16D) The greater emergence of insulin-expressing cells in the 5-Aza-DC treated cultures was accompanied by a drastic decline in the number of glucagon-expressing cells in a concentration-dependent manner (n=3). FIG. 16E) A pooled representation from multiple experiments of the yield of insulin+ cells from untreated (n=3) and demethylated (n=5) T1D-1 DE cells shows that 5-Aza-DC treatment consistently and significantly augments the yield of IPCs by nearly 4-fold, ***p<0.001. Data are represented as mean±SEM.

FIGS. 17A-17B. FIG. 17A) At the end of Stage 4, the yield of Pdx1+ pancreatic progenitor cells from T1D-1 iPS cells was poor (˜12%), which translated into the impaired differentiation of the cells into insulin-expressing cells at the end of Stage 5. To address whether demethylation enhances the yield of Pdx1+ cells from ND iPS cells, we established, in parallel, differentiations of ND iPS cells, treated with or without 10 nM 5-Aza-DC for 18 h on day 4 of differentiation. Demethylation of T1D-1 DE cells corrected this impairment and resulted in >95% Pdx1+ cells at the end of Stage 4 (n=4). However, the yield of Pdx1+ cells was not significantly different between untreated and 5-Aza-DC treated ND IPC cultures. Thus, demethylation enhances the differentiation into IPCs of specifically T1D iPS cells but not ND iPS cells. Undifferentiated iPS cells served as a negative control, whereas the βTC3 mouse insulinoma cell line served as a positive control. FIG. 17B) At the end of Stage 5, the yield of Insulin+ cells from ND iPS cells was 25%, and this yield did not change after treatment with 5-Aza-DC.

FIGS. 18A-18B. Demethylation of ND iPS cells does not significantly enhance the yield of insulin+ cells and Pdx1+ cells. To address whether demethylation enhances the yield of Pdx1+ cells and insulin+ cells from ND iPS cells, in parallel, differentiations of ND iPS cells, treated with or without 10 nM 5-aza-DC for 18 h on day 4 of differentiation (n=2) were established. Controls for staining were iPS cells (negative) and βTC3 mouse insulinoma cells (positive). FIG. 18A) The yield of Pdx1+ cells was not significantly different between untreated and 5-aza-DC treated ND IPC cultures, and this translated into FIG. 18B) equivalent yield of insulin-expressing cells from the two culture conditions. Thus, demethylation enhances the differentiation into IPCs of specifically T1D iPS cells but not ND iPS cells. This is likely because there are specific gene loci on T1D cells that are aberrantly methylated, which is corrected by transient 5-aza-DC treatment.

FIGS. 19A-19B. Demethylation consistently yields a significantly higher yield of IPCs from T1D iPS cells. FIG. 19A) As many as 65% insulin-expressing cells were obtained from T1D iPS cells using this protocol. Controls for staining were iPS cells (negative) and βTC3 mouse insulinoma cells (positive). FIG. 19B) Histogram depiction of these data reveals alignment of the robust insulin+peak of the T1D IPCs with that of the βTC3 cells. The negative peak aligned with that of the undifferentiated iPS cells.

FIGS. 20A-20G. T1D IPCs possess insulin granules and are glucose-responsive. FIG. 20A) IPCs stain positive for cytoplasmic C-peptide, confirming de novo production of insulin, and express Nkx6.1 in the nucleus. Scale bar=10 μm. FIG. 20B) Human islets possess insulin granules of various maturities that are differentiated by the color and shape of the core granule. However, all granules possess a characteristic “halo” surrounding them, which is very specific to the insulin granule. FIG. 20C) Similar to islets, T1D IPCs derived from this protocol possess insulin granules of various maturities, confirming their authenticity and similarity to human islets (n=3 experiments). FIG. 20D) Comparison of the number of granules per cell in islets and IPCs reveals a nonsignificant difference between the two cell types. Data are represented as mean±SEM, n=57 IPCs and 28 islet pancreatic β-cells counted. FIG. 20E-20G) After baseline equilibration in 2.8 mM glucose solution (low glucose or LG), exposure to 28 mM glucose (high glucose or HG) resulted in significant increase in insulin secretion by both IPCs (FIG. 20E) and islets (FIG. 20F). However, the amount of insulin secreted was significantly lower in IPCs than what was observed for human islets. Still, the fold-increase in insulin secretion is higher for IPCs than for islets (FIG. 20G), n=4 (two experiments of duplicates), **p<0.01, ***p<0.001, ****p<0.0001. The variation in cell numbers across the cell batches was normalized by total protein content per well.

FIGS. 21A-21D. T1D IPCs rapidly correct hyperglycemia in diabetic mice. FIG. 21A) STZ-induced diabetic mice (blood glucose levels of ≧300 mg/dL) show rapid correction of hyperglycemia after transplantation with IPCs (n=8). All of the mice show complete and consistent normalization of blood glucose levels within 28 days of IPC transplant. FIG. 21B) When subjected to supraphysiological glucose challenge, T1D IPC-injected mice (showing 5 weeks of stable correction) show effective management of the glucose bolus (2 mg/kg i.p.) by recovering to the baseline normoglycemic state within 4 hours. In contrast, nontransplanted diabetic mice do not recover from severe hyperglycemia. Nondiabetic mice recover to normoglycemia more quickly than IPC-transplanted mice. FIG. 21C) Computation of the “Area Under the Curve” (AUC) for the three treatment groups demonstrates that while IPC-injected mice show superior glucose correction kinetics compared to nontransplanted diabetic mice, they show poorer kinetics compared to nondiabetic control mice. FIG. 21D) STZ-induced diabetic mice (blood glucose levels of ≧300 mg/dL) show rapid correction of hyperglycemia after transplantation with IPCs (n=8). All of the mice show complete and consistent normalization of blood glucose levels within 28 days of IPCs transplant.

FIGS. 22A-22D. T1D IPCs form organoids after transplantation that express insulin. FIG. 22A) Vascularized tissue derived from the T1D iPS cell-derived IPCs 8 weeks post-transplantation reveals the presence of an organoid-like structure. FIG. 22B) H&E staining shows glandular morphology of the cells and pancreatic beta cells similar to cadaveric beta cells. Additionally, the presence of duct-like lumens (marked by yellow arrowheads) was observed around which the cells were organized. Image scale bar=50 μm and inset scale bar=10 μm. FIG. 22C) Immunofluorescence analysis of cryo-embedded sections of this organoid reveals the presence of insulin-expressing cells that do not express glucagon or somatostatin, FIG. 22D) whereas on another section of the organoid, both insulin-expressing and somatostatin-expressing cells were detected. Scale bar=50 μm.

FIGS. 23A-23B. FIGS. 23A and 23B together provide sequences for Activin, Wnt3a, HGF, Excendin-4, KGF, Noggin, GLP, and IGF1.

### DETAILED DESCRIPTION

Type 1 diabetes can be treated by transplanting either the whole pancreas or isolated pancreatic islets. However, there is a chronic shortage of suitable donors. As described herein, iPS cells derived from a patient with Type 1 diabetes (T1D) were used to generate glucose-responsive insulin producing cells (IPCs). The cells responded to high glucose stimulation by secreting insulin in vitro. Their granules were identical to those found in cadaveric β-cells. When transplanted in immunodeficient mice that had developed streptozotocin-induced diabetes, mice achieved normoglycemia within 28 days. None of the mice died or developed teratomas. Because the cells are derived from “self”, immunosuppression is not required, providing a safer treatment option for T1D patients. Additionally, these cells can be used for drug screening, thereby accelerating drug discovery. The approach described here will overcome need to await cadaveric pancreatic tissue.

### Exemplary Methods

In one embodiment, a method to prepare insulin secreting cells from a skin sample from a mammal is provided. The method includes providing a sample of skin cells from a mammal; subjecting the skin cells to conditions that convert the skin cells to induced pluripotent stem cells culturing the induced pluripotent stem cells stepwise under conditions that induce differentiation to definitive endodermal cells, wherein the steps include culturing the cells in a gelatinous protein mixture and optionally applying a demethylation agent; and treating the definitive endodermal cells to a plurality of agents that are sequentially applied which result in stepwise differentiation of the definitive endodermal cells to insulin secreting cells. In one embodiment, the mammal is a human that has type 1 diabetes. In one embodiment, the treating includes differentiating the definitive endodermal cells to posterior foregut cells, differentiating the posterior foregut cells to pancreatic endodermal or progenitor cells, differentiating the pancreatic endodermal or progenitor cells to endocrine precursors, and differentiating the endocrine precursor cells to insulin producing cells. In one embodiment, the definitive endodermal cells are treated with at least one of keratinocyte growth factor (KGF), L-ascorbic acid, or Y27632, or any combination thereof. In one embodiment, the definitive endodermal cells are treated with at least one of a KGF receptor (KGFR) agonist, L-ascorbic acid or an analog thereof or a Rho-associated kinase inhibitor, or any combination thereof. In one embodiment, the posterior foregut cells are treated with at least one of SANT-1, retinoic acid, Noggin, B27, TPB, L-ascorbic acid, or keratinocyte growth factor, or any combination thereof. In one embodiment, the posterior foregut cells are treated with at least one of a Smo inhibitor and Sonic Hedgehog signaling pathway antagonist, retinoic acid or an analog thereof, an inactivator of TGF-beta superfamily signaling proteins, B27, a PKC activator, L-ascorbic acid or an analog thereof, or a KGFR agonist, or any combination thereof. In one embodiment, the pancreatic endodermal or progenitor cells are treated with at least one of ALK5i, Noggin, B27 Supplement, glucagon like peptide-1 (GLP1), SANT1, retinoic acid, DAPT or heparin, or any combination thereof, followed by treatment with ALK5i, Noggin, B27 Supplement, GLP1, DAPT, heparin, or T3, or any combination thereof. In one embodiment, the pancreatic endodermal or progenitor cells are treated with at least one of an inhibitor of TGF-beta RI kinase, an inactivator of TGF-beta superfamily signaling proteins, B27 Supplement, GLP1 or an analog thereof, a Smo inhibitor and antagonist of Sonic Hedgehog signaling, retinoic acid or an analog thereof, an inhibitor of gamma-secretase complex, or heparin or an analog thereof, or any combination thereof, followed by treatment with an inhibitor of TGF-beta RI kinase, an inactivator of TGF-beta superfamily signaling proteins, B27 Supplement, GLP1 or an analog thereof, an inhibitor of gamma-secretase complex, or heparin or an analog thereof, or any combination thereof. In one embodiment, the endocrine precursor cells are treated with at least one of nicotinamide, IGF-1, GLP-1, ALK5i, T3, or heparin, or any combination thereof. In one embodiment, the endocrine precursor cells are treated with at least one of nicotinamide or an analog thereof, IGF-1 or an analog thereof, GLP-1 or an analog thereof, an inhibitor of TGF-beta RI kinase, T3 or an analog thereof, or heparin or an analog thereof, or any combination thereof. In one embodiment, the insulin secreting cells express insulin at levels that are at least 30% that of insulin secreting cells in a mammal that is not diabetic.

In one embodiment, the steps to promote differentiation include treating induced pluripotent stem cells with at least one of Activin A or Wnt3a, or both, thereby providing definitive endodermal cells; treating the definitive endodermal cells with at least Activin A and introducing the treated definitive endodermal cells to a gelatin coated substrate and are treated at least one of keratinocyte growth factor, Noggin, or B27 having insulin but lacking vitamin A, or any combination thereof, thereby providing pancreatic endoderm cells; and treating the pancreatic endoderm cells with at least one of HGF, exendin-4, or nicotinamide, or any combination thereof. In one embodiment, the induced pluripotent stem cells are cultured with at least one of a TGF-beta family member or a Wnt family member, or both, thereby providing definitive endodermal cells. In one embodiment, the definitive endodermal cells are introducing to a gelatin coated substrate and are treated at least one of a keratinocyte growth factor receptor agonist, an inactivator of TGF-beta superfamily member signaling proteins, or B27 having insulin but lacking vitamin A, or any combination thereof, thereby providing pancreatic endoderm cells; wherein the pancreatic endoderm cells are treated with at least one of HGF or an analog thereof, exendin-4 or an analog thereof, or nicotinamide or an analog thereof, or any combination thereof, thereby providing pancreatic endocrine precursors; and wherein the pancreatic endocrine precursors are treated with an inactivator of TGF-beta superfamily member signaling proteins. In one embodiment, the definitive endodermal cells are treated with at least a TGF-beta family member and introducing the treated definitive endodermal cells to a gelatin coated substrate and are treated at least one of a keratinocyte growth factor receptor agonist, an inactivator of TGF-beta superfamily member signaling proteins, or B27 having insulin but lacking vitamin A, or any combination thereof, thereby providing pancreatic endoderm cells; and treating the pancreatic endoderm cells with at least one of HGF or an analog thereof, exendin-4 or an analog thereof, or nicotinamide or an analog thereof, or any combination thereof.

### Exemplary Factors

Factors useful to induce differentiation towards pancreatic beta cells include, but are not limited to, a KGF receptor (KGFR) agonist, L-ascorbic acid or an analog thereof, a Rho-associated kinase inhibitor, a Smo inhibitor and Sonic Hedgehog signaling pathway antagonist, e.g., SANT1, retinoic acid or an analog thereof, an inactivator of TGF-beta superfamily signaling proteins, B27, a PKC activator, e.g., TPB, an inhibitor of TGF-beta RI kinase, e.g., Alk5i, B27 Supplement, GLP1 or an analog thereof, an inhibitor of gamma-secretase complex, e.g., DAPT, heparin or an analog thereof, T3 or an analog thereof, nicotinamide or an analog thereof, IGF-1 or an analog thereof, a TGF-beta family member, a Wnt family member, B27 having insulin but lacking vitamin A, HGF or an analog thereof, or exendin-4 or an analog thereof.

For example, one or more of the following exemplary agents may be employed: a KGF receptor (KGFR) agonist, L-ascorbic acid or an analog thereof, and a Rho-associated kinase inhibitor may include keratinocyte growth factor (KGF), L-ascorbic acid, or Y27632, a Smo inhibitor and Sonic Hedgehog signaling pathway antagonist, retinoic acid or an analog thereof, an inactivator of TGF-beta superfamily signaling proteins, B27, a PKC activator, L-ascorbic acid or an analog thereof, or a KGFR agonist, may include SANT-1, retinoic acid, Noggin, B27, TPB, L-ascorbic acid, or keratinocyte growth factor; and an inhibitor of TGF-beta RI kinase, an inactivator of TGF-beta superfamily signaling proteins, GLP1 or an analog thereof, a Smo inhibitor and antagonist of Sonic Hedgehog signaling, retinoic acid or an analog thereof, may include ALK5i, Noggin, glucagon like peptide-1 (GLP1), SANT1, retinoic acid, DAPT, heparin, or T3. For example, one or more of the following exemplary agents may be employed: keratinocyte growth factor (KGF), L-ascorbic acid, Y27632, SANT-1, retinoic acid, Noggin, B27, TPB, L-ascorbic acid, ALK5i, B27 Supplement, glucagon like peptide-1 (GLP1), DAPT, heparin, T3, nicotinamide, IGF-1, GLP-1, or T3.

Activin and analogs thereof include those having 80%, 85%, 90%, 95%, 98%, or more identity to Accession No. EAW 94139, which is incorporated by reference herein.

Wnt3a and analogs thereof include those having 80%, 85%, 90%, 95%, 98%, or more identity to Accession No. BAB61052 or AAI03924, which are incorporated by reference herein.

HGF and analogs thereof include those having 80%, 85%, 90%, 95% 98%, or more identity to Accession No. BAA14348, AAA64297 or AAA64239, which are incorporated by reference herein.

Exendin-4 and analogs thereof include those having 80%, 85%, 90%, 95% 98%, or more identity to Accession No. P236349 or C6EFG, which are incorporated by reference herein.

KGF and analogs thereof, e.g., palifermin, include those in CA2202390C or CA2201944, or those having 80%, 85%, 90%, 95% 98%, or more identity to Accession No. NP_002000 or NP_032034, which are incorporated by reference herein. Y27632 and analogs thereof include but are not limited to those in Table 1 in Liao et al., J. Cardio. Pharma, 50:17 (2009), which is incorporated by reference herein.

SANT1 and analogs thereof include but are not limited to those in Rominger et al., J. Pharmacol., 329:995 (2009), which is incorporated by reference herein.

Noggin and analogs thereof include those having 80%, 85%, 90%, 95% 98%, or more identity to Accession No. AAA83259, NP_05441, EAW94528, or 34027, which are incorporated by reference herein.

TPB analogs include other PKC activators such as PMA, bryostatin, okadaic acid or benzolactam derived molecules.

GLP and analogs thereof include those having 80%, 85%, 90%, 95% 98%, or more identity to Accession No. NP_002045 (preprotein), which is incorporated by reference herein.

DAPT analogs include inhibitors of gamma secretase other than DAPT, e.g., see Tables 1 and 2 in Olsaukas-Kuprys et al., Onio. Targets Ther., 6:943 (2013), which are incorporated by reference herein.

ALK5 analogs include other inhibitors of TGF-beta RI kinase in addition to Alk5i, see, e.g., Geillebert et al., Bioorg. Med. Chem. Lett., 19:2277 (2009), which is incorporated by reference herein.

Analogs of nicotinamide include but are not limited to those in Sanchez-Pacheco et al, Mol. Cell Endocrin., 91:127 (1993)), which is incorporated by reference herein.

Analogs of T3 include but are not limited to those in Riberio, Thyroid, 18:197 (2008), which is incorporated by reference herein.

Analogs of IGF1 include those having 80%, 85%, 90%, 95% 98% or more identity to Accession No. CAG46659 or AAI48267, which are incorporated by reference herein.

Analogs of heparin include but are not limited to those in Belmiro et al., J. Biol. Chem., 284:11267 (2009), which is incorporated by reference herein.

Analogs of ascorbic acid but are not limited to those Toyada-Ono et al., J. Biosci. Bioeng., 99:361 (2005), which is incorporated by reference herein.

Analogs of retinoic acid include but are not limited to those in Caselli et al., Antivirus Thera., 13:199 (2008), which is incorporated by reference herein.

Non-limiting examples of DNA demethylating (demethylation) agents are 5-aza-2-deoxycytidine (decitabine), 5-azacytidine (azacitidine), zebularine, procaine, RG108, S-5-adenosyl-L-homocysteine, Caffeic acid, Chlorogenic acid, Epogallocatechin gallate, Hydralazine hydrochloride, Procainamide hydrochloride or Psammaplin A. Other cytidine analogues, such as, e.g. Pseudoisocytidine, 5-fluoro-2-deoxycytidine, 5,6-dihydro-5-azacytidine, 2′-deoxy-5,6-dihydro-5-azacytidine, 6-azacytidine, 2′,2′-Difluoro-deoxycytidine (gemcitabine), or Cytosine-beta-D-arabinofurasonide, in particular 5-fluoro-2-deoxycytidine, 5,6-dihydro-5-azacytidine, 2′-deoxy-5,6-dihydro-5-azacytidine, 6-azacytidine or 2′,2′-Difluoro-deoxycytidine (gemcitabine).

Concentrations of the factors and agents range from about 0.05 μM to about 20 μM or about 10 ng/mL to about 20 μg/mL, e.g., about 1 nM to about 50 μM, or about 1 nM to about 1 μM, about 1 nM to about 500 nM, about 1 nM to about 250 nM, 1 nM to about 50 nM, about 0.01 mM to about 5 mM, or about 10 ng/mL to about 300 ng/mL, 1 ug/mL to about 20 μg/mL, or about 10 ng/mL to about 200 ng/mL.

The invention will be described by the following non-limiting examples.

### Example 1

### Materials and Methods

Differentiation of Human iPS Cells into IPCs

Undifferentiated human iPS cells at passage 28 were maintained on irradiated primary mouse embryonic feeder cells until they formed individual colonies. The undifferentiated iPS cell colonies were subjected to a multistep differentiation protocol. Initially, the iPS cells were treated with serum free DMEM/F12 supplemented with Activin A (100 ng/mL) and Wnt3a (25 ng/mL) for 24 hours. Subsequently, the cells were treated with DMEM/F12 supplemented with 100 ng/mL Activin A and 0.2% FBS for 4 days to allow their robust differentiation into definitive endodermal (DE) cells. The DE cells were trypsinized to generate single cell suspension and plated them (3×10⁴ cells/cm²) onto gelatin coated 6 well plates. The cells were maintained in DMEM/F12 supplemented with retinoic acid (2 μM), keratinocyte growth factor (25 ng/mL), Noggin (50 ng/mL), 0.5% ITS, 2% B27 (contained with recombinant insulin) without vitamin A, 1% non-essential amino acids, 1% glutamax and 0.1 mM β-mercaptoethanol for 6 days to generate pancreatic endoderm. The pancreatic endoderm thus generated was cultured in DMEM supplemented with HGF (20 ng/mL), exendin-4 (50 ng/mL), nicotinamide (10 mM) for 6 days to generate pancreatic endocrine precursors. The pancreatic endocrine precursors were allowed to mature and undergo expansion in DMEM supplemented with 10% FBS and nicotinamide (10 mM) for 8-10 days until further characterization or transplantation.

### Mice

All animal experiments were approved and performed according to International Animal Care and Use Committee (IACUC) guidelines. The University of Iowa animal vivarium is accredited by the Association for the Assessment and Accreditation of Laboratory Animal Care (AAAL AC). Eight week-old Rag2^{-/-}γc^{-/-} male mice (Jackson Laboratory, Bar Harbor, Me., USA) were used for the transplantation experiments. Diabetes was induced by five consecutive intraperitoneal streptozotocin (STZ) (EMD Millipore Corporation, Billerica, Mass.<USA) injections (40 mg/Kg body weight). STZ was reconstituted in ice cold fresh sodium citrate buffer (pH 4.5) immediately prior to injection. The fasting blood glucose levels were regularly monitored using a HemoCue glucose 201 analyzer (HemoCue AB, Angelholm, Sweden). Mice with blood glucose levels >350 mg/dl for two consecutive readings that were five days apart were considered diabetic. Diabetic Rag2^{-/-}γc^{-/-} mice do not survive beyond 15-20 days due to severe hyperglycemia. Approximately 5×10⁶ human iPS cell-derived IPCs were transplanted under the kidney capsule of the diabetic mice as described earlier (Raikwar and Zavazava, 2012). The IPC transplanted mice were kept under observation for 150 days and their blood glucose profiles were monitored on weekly intervals.

### Bioluminescence Imaging

The undifferentiated human iPS cells were transfected with pGL4/RIP-Luc vector and their in vitro differentiation into IPCs at various stages was monitored by real-time bioluminescence imaging as described earlier (Raikwar and Zavazava, 2009). The relative Luciferase expression at various stages was calculated and the results were analyzed by GraphPad prism 5.

### Transmission Electron Microscopy

To confirm whether the human iPS cell-derived IPCs are indeed producing insulin, the IPCs were subjected to transmission electron microscopy. To facilitate the identification of IPCs, the cells were subjected to real-time bioluminescence imaging first and the cells displaying the robust bioluminescence signal were considered insulin producing and were processed for transmission electron microscopy. As a positive control we used the human pancreatic islets made available through the City of Hope Integrated Islet Distribution Program. Briefly, the IPCs or the human pancreatic islets were fixed in 2.5% wt/vol glutaraldehyde for 24 hours. The fixed IPCs and the islets were treated with 100 mM cacodylate buffer (pH 7.4) containing 3% wt/vol formaldehyde, 1.5% wt/vol glutaraldehyde for 15 minutes. The fixed IPCs or the pancreatic islets were subjected to osmification in 1% Osmium tetroxide and then stained with Uranyl acetate after washing step. The cells were subsequently dehydrated through a series of graded ethanol solutions and embedded in Epon. The embedded cells were cut into 30 μM thin sections using glass knife on a Leica Ultramicrotome and analyzed on JEOL JEM-1230 Transmission electron microscope. The images were acquired using Gatan Ultrascan CCD camera and the images were analyzed by using Image J program.

### Mitochondria Stress Test

Oxygen consumption rate (OCR) was measured by using an intact-cell respirometer designed for adherent cells (Seahorse Bioscience, North Billerica, Mass.). Human iPS cell-derived IPCs were grown in special 24-well plates designed for respirometer analyses. OCR was determined in assay medium consisting of medium M199 lacking sodium bicarbonate for 60 minutes. Before analysis, IPCs within individual wells were exposed to either, low glucose (2.8 mM), high glucose (20 mM), high glucose (20 mM)+Nifedipine. During respirometry, wells were sequentially injected at the times indicated in the figures with oligomycin (2 μM) to block ATP synthase to assess respiration required for ATP turnover (OCR_{ATP}), carbonyl cyanide p-[trifluoromethoxy]-phenyl-hydrazone (FCCP; 2 μM), a proton ionophore, to induce chemical uncoupling and induce maximal respiration, or antimycin A (0.5 μM) plus rotenone (2 μM) to completely inhibit electron transport and measure non-mitochondrial respiration. The FCCP concentration used in these studies was determined by titration with differing amounts of the uncoupler by using the least amount required for maximal uncoupling in cells unexposed to MTQAs. OCR (pmol per minute per microgram of DNA) was determined as the average number recorded during time periods defined as intervals between the above sequential injections. Basal OCR was determined as respiration before injection of any compounds minus non-mitochondrial OCR. OCR_{ATP} was determined as basal OCR minus OCR after oligomycin injection. OCR accountable by the proton leak was calculated as OCR in the presence of oligomycin minus non-mitochondrial OCR. Maximal uncoupled respiration was calculated as OCR after FCCP minus non-mitochondrial OCR. All values for OCR were normalized to DNA content of the individual wells. ECAR was quantified simply as the recorded acidification rate during the respiratory conditions delineated above.

### Immunostaining and Confocal Microscopy

The human iPS cells grown on chambered glass slides were differentiated into IPCs and were fixed with 2% paraformaldehyde, quenched in PBS containing 30 mM glycine and permeabilized with 0.1% Triton X-100 for 30 minutes at RT. The cells were stained with primary antibodies against Foxa2 (SC-6554, goat polyclonal IgG, Santa Cruz Biotechnology, Santa Cruz, Calif.), Sox17 (SC-17356, goat polyclonal IgG, Santa Cruz Biotechnology, Santa Cruz, Calif.), glucagon (SC-13091, rabbit polyclonal IgG, Santa Cruz Biotechnology, Santa Cruz, Calif.), insulin (SC-7838, goat polyclonal IgG, Santa Cruz Biotechnology, Santa Cruz, Calif.) respectively. The cells were visualized by the use of either the Alexa Fluor 488 conjugated donkey anti-rabbit (A21206, Molecular Probes, Invitrogen, Carlsbad, Calif.) or Alexa Fluor 546 conjugated donkey anti-goat (A11056, Molecular Probes, Invitrogen, Carlsbad, Calif.) secondary antibodies. Multiphoton imaging was performed on Zeiss LSM 710 microscope using 20× objective lens and the images were captured as grayscale pictures and processed using the ZEN 2011 imaging software. Immunohistochemical analysis and hematoxylin-eosin staining of the tissue sections was performed.

### Intra-Peritoneal Glucose Tolerance Test (IPGTT)

IPGTT was performed as we recently described (Raikwar and Zavazava, 2012). Briefly, the control and IPC transplanted mice were fasted overnight for 16 hours. Next morning, the body weights were calculated, their fasting blood glucose values were monitored and each mouse was injected with a glucose solution intraperitoneally at a dose of 2 g/kg body weight. Thereafter, the blood glucose levels were monitored at a regular interval of 30 minutes up to a maximum duration of 180 minutes. The blood glucose values were plotted as a function of time and the incremental area under the curve were calculated.

### Statistical Analysis

The experimental data were analyzed using the GraphPad Prism 5 software (GraphPad Software, Inc., San Diego, Calif., USA). The data were tested for significance with Student's t-test or one-way ANOVA where applicable. In all cases, *P<0.05 was considered significant.

### Results

Human iPS Cells Differentiate into Insulin Producing Cells

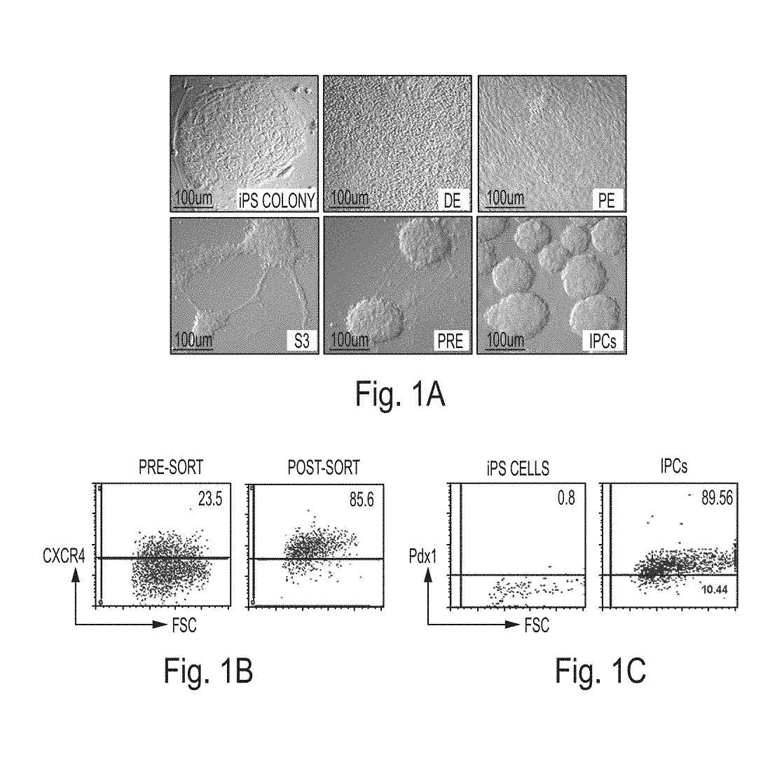

Here, it was asked whether human iPS cells undergo pancreatic lineage commitment to generate IPCs. Human iPS cells were generated as described in Kim et al. (2013). To generate IPCs, a multistep differentiation protocol was used. The endoderm was derived by treating human iPS cells with Activin A (FIG. 1A). The endodermal cells express CXCR4, which was exploited to sort the cells out by immunomagnetic bead separation (FIG. 1B). The definitive endodermal cells were allowed to further differentiate and undergo expansion into Pdx1-expressing pancreatic endodermal (DE) cells in the presence of retinoic acid and KGF. Intracellular staining revealed that 89.56% of the pancreatic endodermal (PE) cells were positive for Pdx1 (FIG. 1C). The pancreatic endodermal cells were further cultivated in the presence of HGF and Exendin 4 to generate endocrine progenitors which spontaneously gave rise to three dimensional pancreatic islet like clusters (FIG. 1A). During intermediate stages Pre1 and Pre2, the cells formed projections similar to those of neuronal cells. The morphology of the cells changed during the differentiation procedure. With time the cells transitioned into pancreatic precursors that were polymorphic and flat. Finally, the cells formed cell clusters by day 25. At this stage we termed them IPCs.

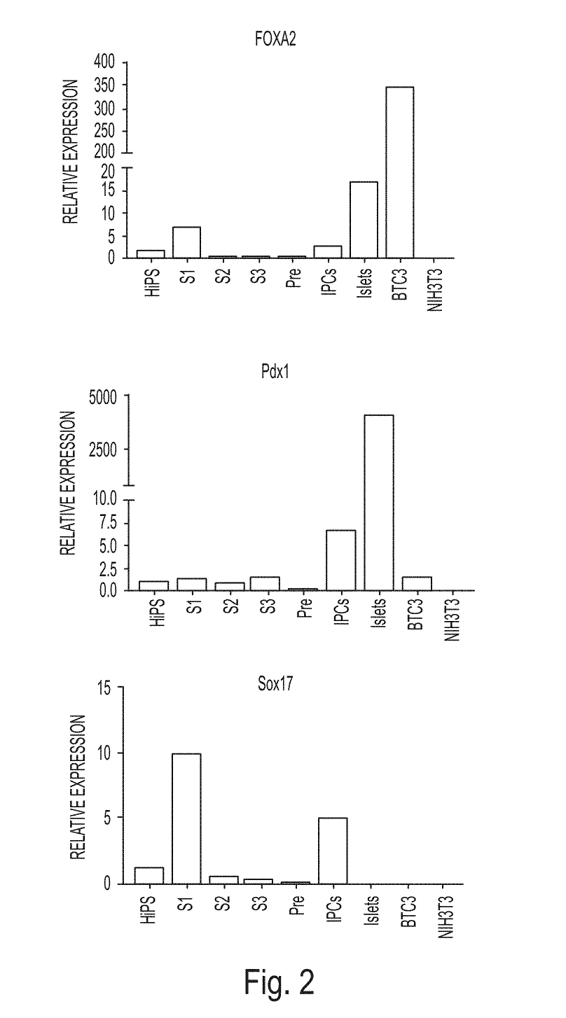

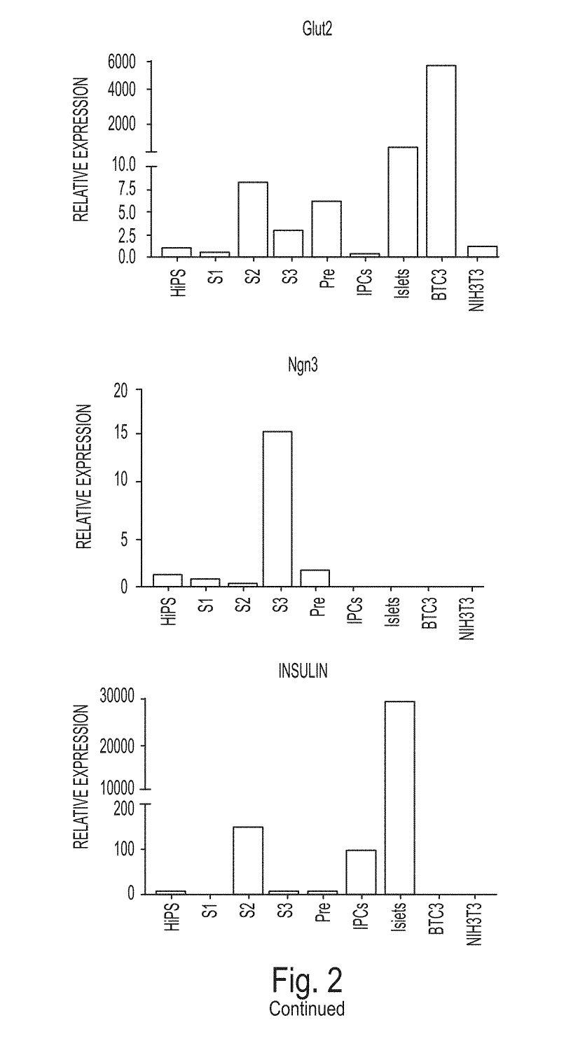

To confirm that the cells were pancreatic, we used real time quantitative PCR to study gene expression during the differentiation process. The present result suggest that during the definitive endoderm formation there is an upregulation of Sox17 and Foxa2 (FIG. 2). As expected the cells undergoing differentiation transiently expressed high levels of Ngn3. The differentiated cells expressed Pdx1, insulin as well as Glut 2. The human iPS cell-derived IPCs revealed the expression of Foxa2 which has been shown earlier to be important not only for pancreatic beta cell development but also for insulin secretion. The fully differentiated IPCs expressed insulin which was, however, much lower than that detected in fresh human pancreatic islets.

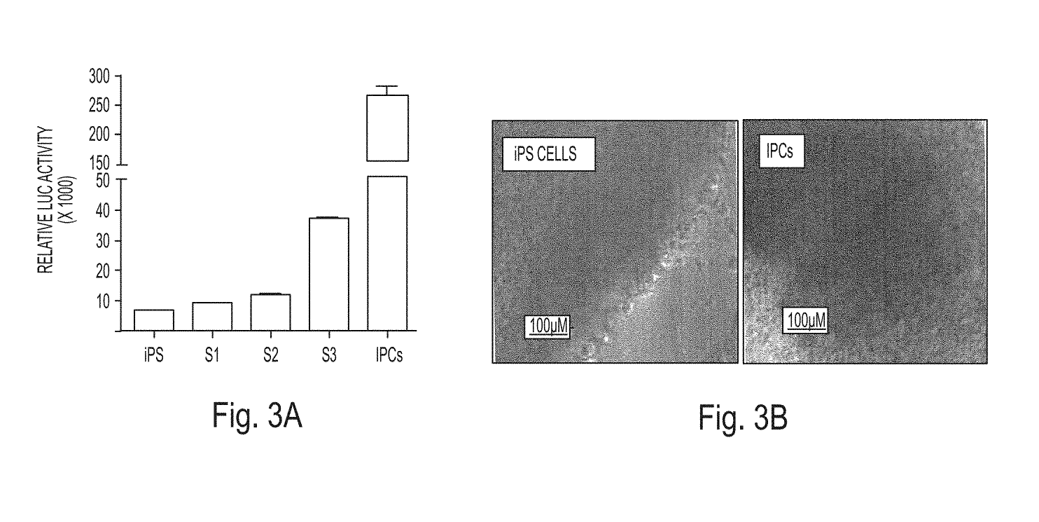

To monitor IPC differentiation in vitro in real-time, the undifferentiated human iPS cells were transfected with a vector expressing RIP-Luc. There was no detectable luciferase expression either in definitive endodermal cells or pancreatic endodermal cells. However, differentiation of pancreatic endodermal cells into endocrine progenitors was marked by detectable luciferase expression which increased significantly in the IPCs (FIG. 3A). Progressive differentiation of human iPS cells leads to transcriptional activation of the rat insulin promoter (RIP), which results in significantly enhanced luciferase expression in the IPCs, suggesting secretion of insulin. To further confirm these results, the IPCs were stained with dithizone, which is a sulfur containing compound which binds to metals, including zinc which is found in pancreatic β-cells. The IPCs stained bright red suggesting that they contained zinc (FIG. 3F).

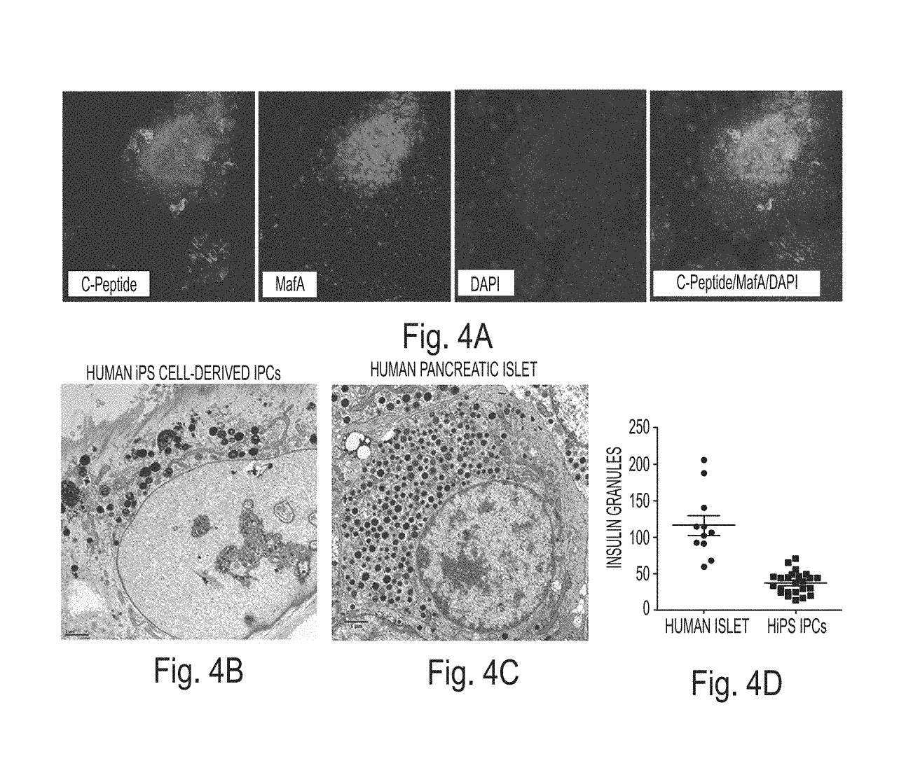

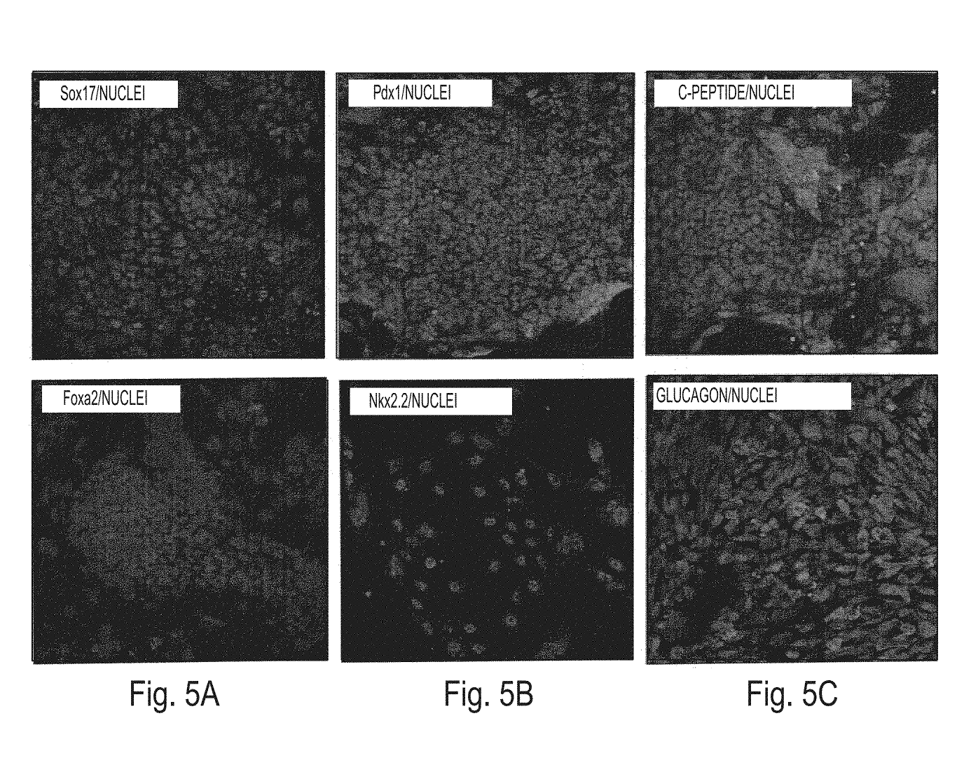

The cells were immune-stained for a number of pancreatic transcription factors including C-peptide, Maf A and glucagon (FIG. 4A, FIG. 5A). Insulin was not stained for the culture medium contained recombinant insulin. The data show that the cells stained positive for Sox17, Pdx1, C-peptide, Foxa2, Nkx2.2 and glucagon (FIG. 5). To further characterize the IPCs, transmission electron microscopy of the IPC clusters was performed. IPCs were compared to fresh human pancreatic islets (FIGS. 4B-C). IPCs showed electron dense insulin secretory granules characteristic of pancreatic beta cells (FIG. 4D). As expected, IPCs expressed far less granules as compared to β-cells. On average, each IPC contained approximately 20-70 insulin secretory granules compared to 50-200 counted in pancreatic islets. In addition, the insulin secretory granules in the IPCs lacked a characteristic halo, which is typically seen in pancreatic beta cells. This observation potentially suggests that the IPCs are immature at this stage. In addition to the granules, the cells also contained multiple mitochondria indicating an active metabolic phenotype.

IPC Mitochondria Consume Oxygen after Stimulation

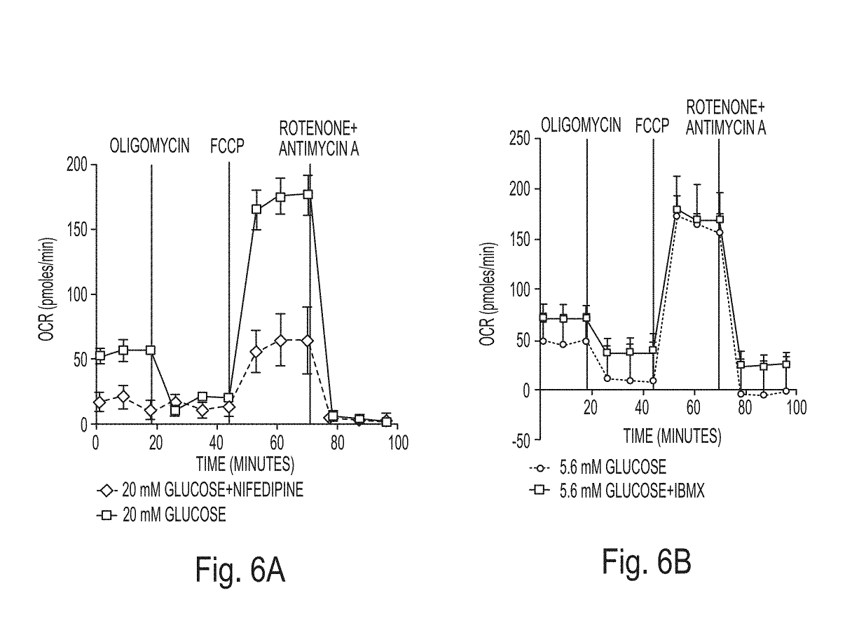

To study the respiration of IPCs, the Mitochondria stress test was used. In high glucose (20 mM), the IPCs significantly increased their oxygen consumption (FIG. 6A). As expected, the calcium channel blocker, Nifedipine, successfully inhibited oxygen consumption. Conversely, in low glucose, IPCs consumed oxygen, which could not be elevated by addition of IBMX (FIG. 6B). These results suggest that human iPS cell-derived HPCs respond to glucose levels.

### Human iPS Cell-Derived IPCs Correct Hyperglycemia in Diabetic Mice

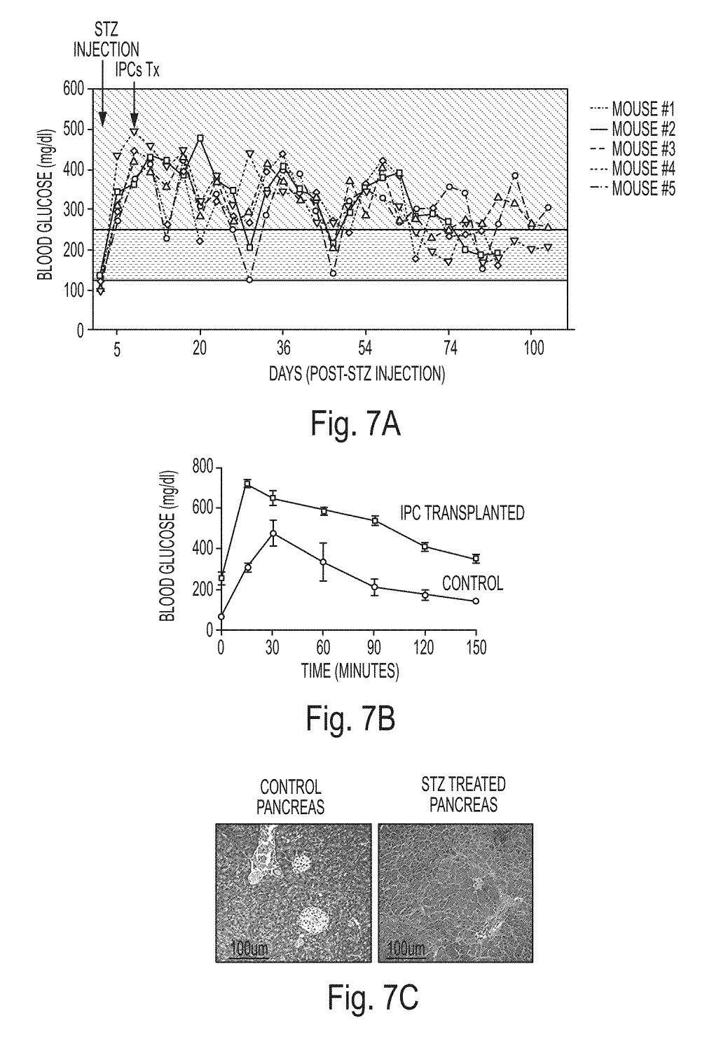

Next, it was asked whether the IPCs correct hyperglycemia in diabetic mice. Diabetic Rag2^{-/-}γc^{-/-} mice were chosen as recipients of IPCs because they lack a functional immune system. 5×10⁶ IPCs were transplanted under the kidney capsule of STZ induced-diabetic Rag2^{-/-}γc^{-/-} mice. The fasting blood glucose levels were monitored regularly over a period of >100 days. The pretransplant blood glucose levels in the STZ induced-diabetic mice were in the range of 400-500 mg/dl. Following IPC transplantation, the fasting blood glucose levels were <200 mg/dl after 100 days in 3 of 6 mice (FIG. 7A). Two mice out of 6 had borderline glucose levels and one mouse died soon after transplantation, and was eliminated from our evaluation. Overall, IPCs effectively regulated glucose levels in all mice. Most of the IPC transplanted mice showed a peak and trough pattern of blood glucose levels indicating that the insulin secretion by the transplanted IPCs may not be fully mature β-cells. These initial results were, however, encouraging. The mice transplanted with IPCs were further subjected to an intra-peritoneal glucose tolerance test at day 100. As compared to the healthy nondiabetic control mice the IPC transplanted mice displayed a poorer blood glucose clearance as compared to control mice (FIG. 7B). The incremental areas under the curve between the control and the IPC transplanted mice were statistically significant, p<0.001, as represented in FIG. 7C. Overall the transplantation data suggest that human iPS cell-derived IPCs are able to regulate hyperglycemia in diabetic mice, however, they are less effective than the pancreas. Further, long-term experiments are required to determine whether the cells continue to secrete insulin over a more extended period than the 150 days studied here and whether their control of hyperglycemia improves with time. As expected, none of the mice developed teratomas.

To rule out that despite streptozotocin treatment the pancreas could have recovered, compromising the IPC results, we histologically examined the pancreas of transplanted mice. Clearly, the pancreas of streptozotocin treated mice was void of any islets as compared to those of control mice (FIG. 7C).

### Real-Time Noninvasive Imaging of Transplanted IPCs by MRI

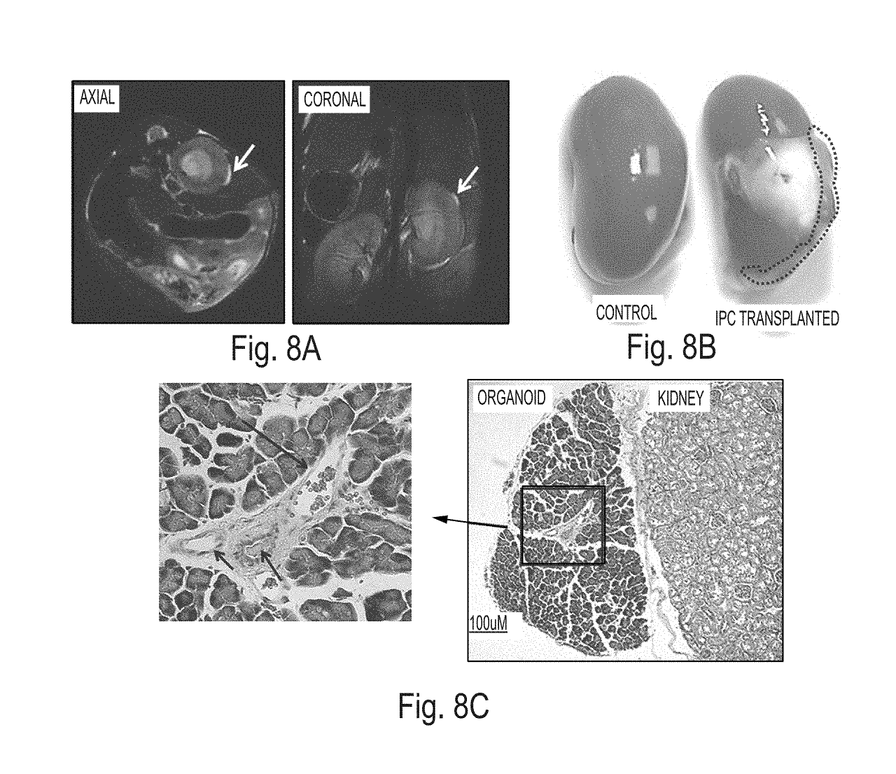

One of the major caveats in the field of islet transplantation is the lack of noninvasive imaging to monitor the fate and function of the transplanted IPCs. Here, it was tested whether MRI can be used to monitor the long term fate of the transplanted IPCs. MRI imaging was performed 150 days post transplantation. The IPCs transplanted under the kidney capsule could easily be identified by MRI as a dense white mass present on the dorsal surface of the kidney on both the coronal as well as axial projections (FIG. 8A). To exclude the possibility of teratoma formation post-IPC transplantation, the mice were sacrificed and both of the kidneys examined. The transplanted IPC mass appeared as a vascularized triangular area (FIG. 8B). 12 mice were transplanted with IPCs and 10 remained untreated with IPCs but were treated with streptozotocin. These control mice died within 10 days. Besides the IPC transplanted mass, there was an area that showed adhesion to the liver, as a result of the surgical procedure, cells were transplanted in the right kidney. Gross examination of the kidneys as well as other internal organs revealed the absence of any teratomas. These data confirm that the IPC-derived organoids can non-invasively be monitored by MRI. Further, the data also revealed that the transplantation of human iPS cell-derived IPCs did not lead to teratoma formation thereby highlighting the safety of the human iPS cell-derived IPCs.

To further examine the pancreatic organoids, the explanted kidneys were stained by H & E. As shown in FIG. 8C, the organoid can be seen next to the kidney but separated by a thin layer of connective tissue. A closer examination further reveals that the organoid had its own blood vessels (insert), suggesting that either the IPCs secreted VEGF or they induced secretion of VEGF by other cells.

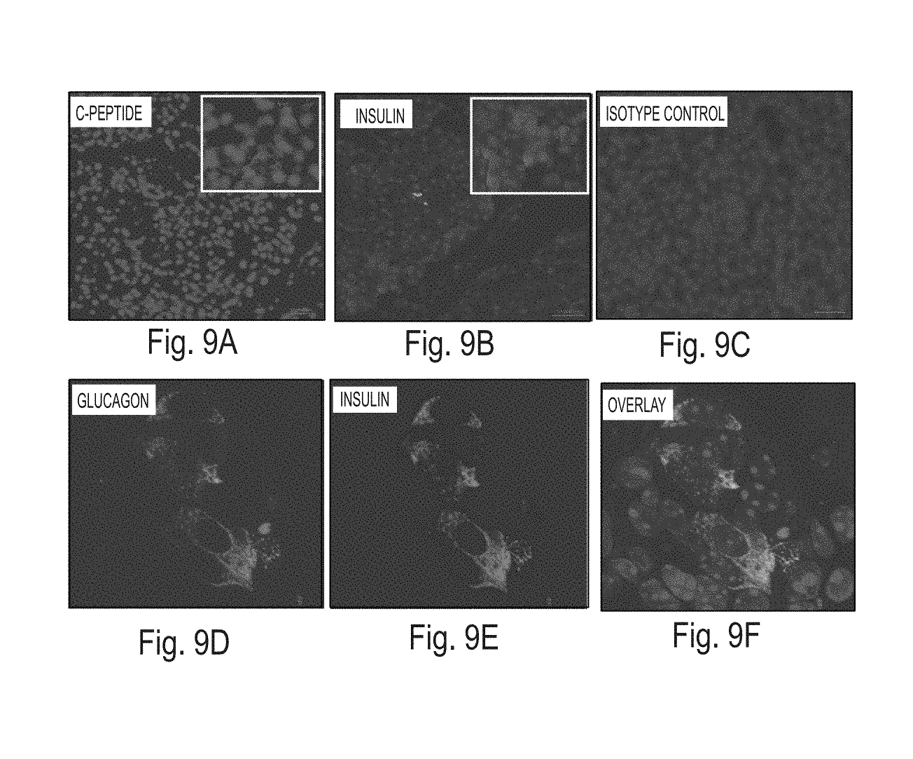

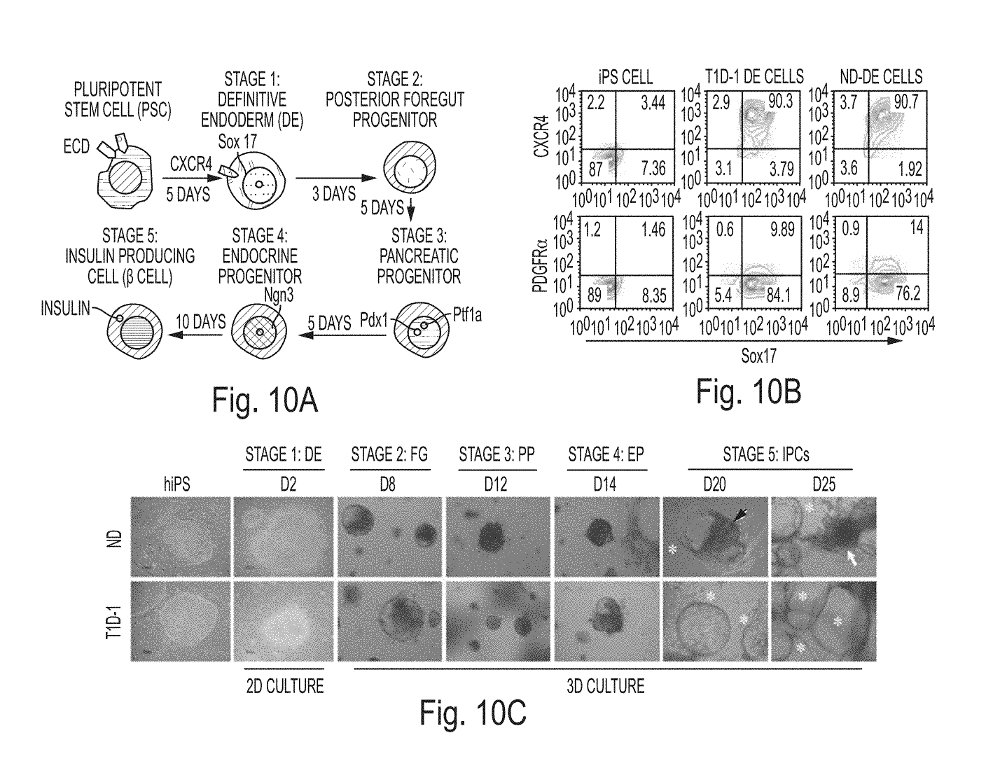

To confirm that the organoid was pancreatic, histological sections of the explanted kidneys were stained for glucagon and insulin (FIG. 9A). In both cases, the stains were patchy, again stressing the fact that even after 150 days, not all cells were β-cell like. Immature n-cells are bihormonal, secreting both insulin and glucagon. This was highlighted in the overlays that showed evidence of secretion of both hormones. Lastly the sections were stained for C-peptide, which stained in a similar pattern (FIG. 9B). Thus, it was shown that after 150 days in the kidney capsule, the iPS cell derived cells continued to be pancreatic.

### Discussion

The ability to reprogram somatic cells into pluripotent stem cells is a very appealing approach with the potential to revolutionize future cell-based therapies (Takahashi et al., 2007; Takahashi and Yamanaka 2006). Here, it was shown that human iPS cells efficiently generate CXCR4-expressing endodermal cells. By following a step by step approach, pancreatic precursor cells were generated that were Pdx1⁺, a master transcription factor that regulates the development of the pancreas (Stoffers et al., 1997). Different approaches for generating IPCs have been tried with mixed results. For example, mouse ES cells were directed towards IPCs, but were never able to eliminate partially differentiated cells. Consequently some of the mice that were transplanted with these cells developed teratomas (Raikwar and Zavazava, 2012). In some cases the process was barely efficient in generating IPCs. Here, the IPCs formed cell clusters at the end of the differentiation process. These cell aggregates could be counted and transplanted into diabetic mice.

The gene expression of the differentiating cells was followed by quantitative real time PCR. IPCs clearly had elevated insulin, Sox17 and Pdx1. The levels of Glut2 were quite low, suggesting that the IPCs may still not be fully mature and might poorly respond to glucose challenge. In comparison to pancreatic islets, the level of insulin in the IPCs was relatively low. Indeed when we performed transmission electron microscopy of the IPCs and human islets, IPCs contained only a third of the zinc containing granules compared to those found in islets. These data suggested that freshly differentiated IPCs poorly secrete insulin. These IPCs, however, underwent mitochondrial stress testing showing a good response to high glucose levels, which could be blocked by Nifedipine. Thus, while the cells at this stage were not as robust as pancreatic islets, they responded to glucose.

Interestingly, diabetic mice responded well to the transplantation of IPCs. It is worth noting that after IPC transplantation, the glucose levels were going down and rebounding up again. It is not clear whether this reflects the immaturity of our cells and their poor response to glucose or whether some other metabolic control mechanisms are involved. It was ruled out the possibility that the peaks could have been caused by eating times when the mice were feeding, because mice were fasted before the glucose levels were measured. Others have noted the same phenomenon with ES cell-derived IPCs (Rezania et al., 2012). However, after mice became normoglycemic by day 100, glucose levels showed a steady level with no rebounds. It was hypothesized that the cells had matured and were better able to regulate glucose levels at this stage. The glucose tolerance test showed a gap between control mice and mice that received IPCs. Control mice more efficiently controlled glucose levels. The histological data on the pancreas of the transplanted mice confirmed that streptozotocin had in fact destroyed the mouse's own β-cells. Thus, serum glucose levels of the transplanted diabetic mice can be attributed to the IPCs.

It was surprising to discover in FIG. 5C that IPCs had formed 3D vascularized organoids. The most likely scenario is that the cells secrete VEGF and perhaps other growth factors that promote vascularization. The kidney looked intact and nicely separated from the organoid which had acquired a thin capsule. This may be the first pancreatic organoid to be generated from human IPS cells and is able to regulate serum glucose levels. The kidney capsule has been used by many other researchers for the transplantation of human pancreatic islets (Raikwar and Zavazava, 2011; Luo et al., 2013) and has proven to be an excellent site for the transplantation of islets to treat diabetes. In the present case, IPCs seemed to form an independent organoid with its own blood vessels. To further characterize these new tissues, we need to further determine whether there are neo-ducts in the tissue. Further, DNA array data could provide invaluable data on the gene expression pattern of these new tissues.

The new organoids stained positive for insulin and glucagon. Thus, cells were established that produce endocrine hormones. FIG. 6F shows that the cells appear to be bihormonal, a characteristic of immature pancreatic cells. We recognize that these stains were patchy in the tissue and that the intensity varied, supporting the idea that some cells were more advanced in their maturation and others were not. These data are a first step towards iPS cell-derived IPCs. The protocol needs to further improve towards generating cells that secrete insulin only. The present data are consistent with published data, which suggest that IPCs appear to mature post-transplantation in the in vivo environment (Raikwar and Zavazava, 2011; Guo et al., 2013; Schulz et al., 2012). Others have supplemented the transplantation of IPCs inserting insulin chips in the recipient mice early post transplantation (Rezania et al., 2012). It is unclear whether exogenous insulin contributes to the maturation of IPCs. Although the maturation of IPCs remains a challenge, we are encouraged by the ability to generate an organoid that is fully vascularized and controls serum glucose levels.

Recently, another protocol was reported which led to the generation of IPCs that corrected hyperglycemia in both mice and rats (Rezania et al., 2012). Schulz et al. showed that ES cell-derived IPCs could be produced on a large scale (Schulz et al., 2012). Both reports show that the IPCs mature in vivo after 4-5 months. This is consistent with the present data. However, it is exciting that we created a 3D organoid that secretes insulin in vivo for the first time. This data encourage further advances in this field which could ultimately lead to a therapeutically applicable protocol. More recently it was shown that human IPCs generated from iPS cells required a shorter time than previously reported for IPCs to correct hyperglycemia (Pagliuca et al., 2014; Rezania et al., 2014).

### Example 2

### Methods

Differentiation of Human iPS Cells into Insulin Producing Cells In Vitro

The differentiation of human iPS cells into IPCs lasted 27 days and was performed by driving cells through five stages of differentiation, each with its own set of media cocktails, listed in Table 1. The cell culture media was changed for the cells every day and the media prepared fresh every day. Small molecules and growth factors (ordering information for which is listed in Table 2) were supplemented into warm base media immediately prior to media changes in a dim-light hood.

TABLE 1
Differentiation timeline and media constituent information

| | |
|---|---|
| STAGE 1: | Media 0: (D 0 → D 1) |
| Definitive | DE Base Media + Supplement A + Supplement B |
| Endoderm | |
| D 0 → D 5 | Media 1: (D 1 → D 2, D 2 → D 3, D 3 → D 4, D 4 → |
| | D 5) DE Base Media + Supplement B |
| STAGE 2: | Media 2: (D 5 → D 6, D 6 → D 7) |
| Posterior | DMEM/F-12 + 2% Hyclone FBS + KGF (50 ng/ml) + |
| Foregut | L-Ascorbic Acid (0.25 mM) + Y27632 (10 μM) |
| D 5 → D 8 | |
| | Media 3: (D 7 → D 8) DMEM/F-12 + 2% Hyclone |
| | FBS + KGF (50 ng/ml) + L-Ascorbic Acid (0.25 |
| | mM) |
| STAGE 3: | Media 4: (D 8 → D 9, D 9 → D 10, D 10 → D 11, |
| Pancreatic | D 11 → D 12) |
| Endoderm/ | DMEM-HG + SANT-1 (0.25 μM) + Retinoic acid (2 |
| Progenitors | μM) + Noggin (100 ng/mL) + 1% (vol/vol) B27 + |
| D 8 → D 12 | TPB (50 nM) + L-Ascorbic Acid (0.25 mM) + KGF |
| | (50 ng/ml) |
| STAGE 4: | Media 5: (D 12 → D 13, D 13 → D 14, D 14 → D 15, |
| Endocrine | D 15 → D 16) |
| Precursors | DMEM-HG + ALK5i (10 μM) + Noggin (100 ng/ml) + |
| D 12 → D 17 | 1% (vol/vol) B27 Supplement + GLP-1 (100 nM) + |
| | SANT-1 (0.25 μM) + Retinoic acid (100 nM) + |
| | DAPT (10 μM) + Heparin (0.25 mM) + T3 (1 μM) |
| | Media 6: (D 16 → D 17) DMEM-HG + ALK5i (10 |
| | μM) + Noggin (100 ng/mL) + 1% (vol/vol) B27 |
| | Supplement + GLP-1 (100 nM) + DAPT (10 μM) + |
| | Heparin (10 μg/ml) + T3 (1 μM) |
| STAGE 5: | Media 7: (D 17 → D 27) |
| Insulin | DMEM-HG + 10% HyClone FBS + Nicotinamide (5 |
| Producing | mM) + IGF-1 (10 nM) + GLP-1 (100 nM) + ALK5i |
| Cells | (10 μM) + T3 (1 μM) + Heparin (10 μg/ml) |
| D 17 → | |
| D 27 | |

TABLE 2
Information for differentiation supplements

| Compound | Type | Company | Catalog # | Size | |
|---|---|---|---|---|---|
| 5-aza-2′- | 97% HPLC | Sigma- | A3656-5MG | 5 | mg |
| deoxy- | | Aldrich | | | |
| cytidine | | | | | |
| Nodal | Recombinant | R&D | 3218-ND- | 25 | μg |
| | Human | Systems | 025 | | |
| Wnt3A | Recombinant | R&D | 5036-WN- | 10 | μg |
| | Human | Systems | 010 | | |
| KGF (FGF-7) | Recombinant | Peprotech | 100-19 | 10 | μg |
| | Human | | | | |
| SANT-1 | 98% HPLC | Sigma- | S4572-5MG | 5 | mg |
| | | Aldrich | | | |
| Retinoic | 98% HPLC | Sigma- | R2625- | 100 | mg |
| acid | | Aldrich | 100MG | | |
| Noggin | Recombinant | Peprotech | 120-10C | 100 | μg |
| | Human | | | | |
| GLP-1 | Recombinant | Sigma- | G3265- | 1 | mg |
| | Human | Aldrich | .1MG | | |
| ALK5 | N/A | Enzo | ALX-270- | 5 | mg |
| inhibitor II | | | 445-M005 | | |
| TPB | α-Amyloid | EMD | 565740- | 1 | mg |
| | Precursor | Chemicals | 1MG | | |
| | Protein | | | | |
| | Modulator | | | | |
| IGF-1 | Recombinant | Promega | G5111 | 25 | μg |
| | Human | | | | |
| Nicotinamide | Cell culture | Sigma- | N0636-100G | 100 | g |
| | tested | Aldrich | | | |
| B27 | Without | Invitrogen | 12587-010 | 10 | mL |
| | Vitamin A | | | | |
| L-Ascorbic | Dry powder | Fisher | A61-25 | 25 | g |
| Acid (25° C.) | | Scientific | | | |
| DAPT (4° C.) | N/A | Tocris | 2634 | 10 | mg |
| | | Biosciences | | | |
| Heparin | Cell culture | Sigma- | H3149- | 25 | KU |
| | tested | Aldrich | 25KU | | |
| T3 (3,3′,5- | Cell culture | Sigma- | T6397- | 100 | mg |
| Triiodo-L- | tested | Aldrich | 100MG | | |
| thyronine | | | | | |
| sodium salt) | | | | | |

To initiate the differentiation of iPS cells into DE cells, they were first maintained in the STEMdiff™ Definitive Endoderm Kit (05110, Stem Cell Technologies, Vancouver, BC) for 5 days, while cultured on feeder cells as colonies. After confirmation on Day 5 that the culture contained >90% CXCR4⁺Sox17⁺ cells, the rest of the DE cells were harvested and 3D differentiation was initiated with Media 2. On the day prior to initiating 3D differentiation, matrigel (354277, Corning Inc., Tewksbury Mass.) was thawed on ice overnight in a 4° C. refrigerator. If demethylation of the cells was performed, this was done according to details provided. On the day of 3D differentiation (D5), a 1:1 (vol/vol) mixture of liquid matrigel was mixed with cold DMEM/F-12. Then, in a 24 well plate, 500 μL of the 1:1 mixture was deposited in each well. The plate was replaced at 37° C. for 3 hours to allow the matrigel to solidify sufficiently.

2.5 hours into the incubation, the DE cells were harvested via cell scraping and suspended in warm Media 2. This cell suspension was distributed on top of the matrigel, with each well thus containing 500 μL of the matrigel mixture and 500 μL of the cell suspension. Typically, transferred two wells of DE cells cultured in a 6 well plate into one well of a 24 well plate containing matrigel.

### Glucose Stimulated Insulin Secretion (GSIS) Assay

Static glucose stimulated insulin secretion (GSIS) assays were performed in order to determine the glucose-responsiveness of IPC clusters (Day 27-30 of culture) as compared to human islets (supplied by the IIDP). Standard IIDP standard operating procedures were followed (see below), and the Human Ultrasensitive Insulin ELISA (80-INSHUU-E01.1, ALPCO Diagnostics, Salem, N.H.) was utilized according to manufacturer's instructions for quantitation of insulin in the supernatants. The amount of insulin produced was normalized by the total protein, which was calculated via the Bradford Assay of the cell lysates.

Following IIDP standard operating procedures, Krebs buffer stock solution was prepared as follows by combining the following in a 500 mL flask: 2.98 g HEPES power (25 mM), 3.36 g NaCl (115 mM), 1.01 g NaHCO₃ (24 mM), 0.1864 g KCl (5 mM), 0.1017 g MgCl₂.6H₂O (1 mM), 0.5 g BSA (0.1%). These powders were stirred in deionized water so that the total volume was 500 mL and stirred until dissolved. Subsequently, 0.183 g CaCl₂.2H₂O (2.5 mM) was added and pH of the solution was adjusted to 7.4. After mixing thoroughly, the mixture was filter-sterilized through a 0.22 μm bottle top filter into a sterile bottle and stored at 4° C. until expiration at 4 weeks post-preparation. 280 mM glucose solution was prepared by adding 2.5 g of D-(+)-Glucose (Catalog Number: G5767, Sigma-Aldrich, St. Louis, Mo.) to 50 mL of Krebs buffer stock solution. This mixture was filter sterilized and stored at 4° C. until expiration at 4 weeks post-preparation. On the day of the GSIS assay, 30 mL of a 28 mM (“high glucose”) stock solution was prepared by making a 1:10 dilution of the 280 mM glucose stock solution using Krebs buffer stock solution. Additionally, 30 mL of a 2.8 mM (“low glucose”) stock solution was prepared by making a 1:10 dilution of the 28 mM glucose stock solution using Krebs buffer stock solution. Finally, for KCl polarization challenge assessment, 30 mL of a 30 mM KCl solution was prepared by mixing 22.2 mg KCl in 10 mL of 2.8 mM (“low glucose”) stock solution. These sterile diluted solutions were stored at 4° C. until expiration at 1 week post-preparation, or warmed and equilibrated to 37° C. if used the same day.

Differentiated IPCs (Day 27-30 of culture) from 1 well (about 300 clusters) or human islets (approximately 200 IEQ) were sampled. After washing the cell clusters twice in 1 mL 2.8 mM (“low glucose” or LG), the clusters were resuspended in LG solution and divided into duplicate wells of a 96-well plate. The cells were then preincubated at 37° C. in 200 μL/well of LG solution for 2 hours to bring cells to remove residual insulin and bring cells to a common baseline. The plate was very gently centrifuged and the supernatant discarded. The pelleted cells on the plate were resuspended in 200 μL/well of fresh, equilibrated LG buffer. The plate was placed at 37° C. and the cells were allowed to incubate in LG solution for 1 hour. The plate was very gently centrifuged and the supernatant collected into separate duplicate Eppendorf tubes for future analysis by ELISA (low glucose samples). The pelleted cell clusters were resuspended in 200 μL/well of fresh, equilibrated 28 mM (“high glucose” or HG) buffer. The plate was placed at 37° C. and the cells were allowed to incubate in HG solution for 1 hour. The plate was very gently centrifuged and the supernatant collected into separate duplicate Eppendorf tubes for future analysis by ELISA (high glucose samples). Finally, the pelleted cells were resuspended in 200 μL/well of fresh, equilibrated 30 mM KCl in LG buffer (polarization challenge) for 30 min to release all residual insulin in the cells. The plate was very gently centrifuged and the supernatant collected into separate duplicate Eppendorf tubes for future analysis by ELISA (KCL polarization challenge samples). If not analyzed by ELISA immediately, the supernatants were stored at −80° C.

The cell clusters were then resuspended in PBS, removed from the plate and pelleted in separate Eppendorf tubes in order to assess total protein content as a means of normalizing insulin production across samples. The cell cluster pellets were lysed by resuspension in RIPA lysis buffer (Catalog Number: 20-188, EMD Millipore, Billerica, Mass.) supplemented with a protease inhibitor cocktail (Catalog Number: 11836170001, Roche, Indianapolis, Ind.). The cells were also dissociated using a 30 G needle and syringe apparatus to break cell membranes. After incubating on ice for 30 minutes, the cell clusters were centrifuged at 14,000 rpm for 20 minutes at 4° C. The supernatant containing protein was collected and placed into separate Eppendorf tubes for immediate quantitation by Bradford Assay analysis, which was measured at an O.D. of 595 nm using a BioTek μQuant™ spectrophotometer.

On the day of ELISA, supernatant samples were thawed on ice while the ELISA kit components were brought to room temperature. The volume of each sample (generally about 200 μL) was recorded and the samples were processed using the Human Ultrasensitive Insulin ELISA (Catalog Number: 80-INSHUU-E01.1, ALPCO Diagnostics, Salem, N.H.) according to manufacturer's instructions. The samples were quantitated by a BioTek μQuant™ spectrophotometer at an O.D. of 450 nm. Based on a standard curve, a quadratic equation was derived correlating the amount of insulin in a standard sample to the O.D. Using this equation, the amount of insulin in a test sample (μIU/mL) was calculated and tabulated. The amount of insulin produced was normalized by the total protein in each sample, which was calculated as described above via the Bradford Assay of the lysate generated from the cell clusters.

### Human iPS Cell Lines and Culture Conditions

Two human iPS cell lines were utilized in this study. GM23226 (ND human iPS cells) and GM23262 (T1D human iPS cells) were purchased from the Coriell Institute for Medical Research (Camden, N.J.). These human iPS cells were grown on irradiated Mouse Embryonic Feeder (MEF)-coated (Catalog Number: GSC-6001G, Global Stem, Gaithersburg, Md.) 6-well plates in culture medium containing Dulbecco's modified Eagle's medium/F-12 (DMEM/F-12) supplemented with 20% KnockOut Serum Replacement (Catalog Number: 10828-028, Invitrogen, Grand Island, N.Y.), 50 μg/mL penicillin, 50 μg/mL streptomycin, 1 mM GlutaMAX, 1×NEAA, 100 μM 2-mercaptoethanol (Sigma-Aldrich, St. Louis, Mo.), and 10 ng/mL basic Fibroblast Growth Factor (bFGF, Catalog Number: PHG0261, Invitrogen, Grand Island, N.Y.). Unless otherwise noted, all cell culture reagents were purchased from Invitrogen (Grand Island, N.Y.). Cells were incubated at 37° C. in a 5% CO₂ humid atmosphere. The cells were maintained in their undifferentiated state through daily media changes and were passaged every 5-7 days.

### Demethylation of iPS Cells

5-aza-2′-deoxycytidine (5-aza-DC, Catalog Number: A3656-5MG, Sigma-Aldrich, St. Louis, Mo.)) was used to transiently demethylate iPS cells at concentrations of 1 nM and 10 nM, the latter of which allowed for enhanced cell viability. These concentrations were selected after a thorough screen of concentrations (1 nM, 10 nM, 100 nM, 1 and 10 μM) that could be used to induce demethylation while maintaining cell viability.

After completing 4 days of differentiation in DE differentiation media, the cells were treated with fresh media supplemented with 5-aza-DC. The 5-aza-DC was treated for 18 hours, which spanned the last day of DE differentiation, before being washed with warm DMEM F/12 three times and harvested as described in the next section for initiating 3D differentiation into pancreatic precursor cells.

Because of the highly unstable nature of 5-aza-DC, it was rapidly aliquoted and stored to preserve its effectiveness. Prelabeled Eppendorf tubes were kept at −20° C. to keep them cold, and any 15 mL conical tubes were kept on ice. To dissolve 5-aza-DC, first a 100 mM superstock solution was prepared by adding 219 μL of DMSO to 5 mg (21.9 micromole) of 5-aza-DC. After vortexing the solution, a 1:10 dilution with a final concentration of 10 mM was prepared by adding 1970 μL of sterile ultrapure water. This mixture was filter-sterilized using a chilled 0.22 μM mesh attached to a cold 3 mL syringe. 250 μL aliquots of the 10 mM superstock were frozen in large Eppendorfs for later dilution. A separate fraction of the 10 mM superstock was diluted in 2250 μL of sterile ultrapure water to yield a 1 mM superstock, of which 25 μL were distributed to ˜100 chilled small Eppendorf tubes and frozen immediately at ˜80° C. On the day of demethylation treatment, a vial of 1 mM 5-aza-DC was thawed on ice and diluted 1:100 in cold sterile ultrapure water to yield a 10 μM stock solution that is finally ready for treatment.

1 μL of the 10 μM stock per 1 mL of differentiation media was used to create a final concentration of 10 nM 5-aza-DC, whereas 0.1 μL of the 10 μM stock per 1 mL of differentiation media was used to create a final concentration of 10 nM 5-aza-DC.

### Flow Cytometry

For all flow cytometry experiments, undifferentiated iPS cells were used as negative controls for staining, and human islets (supplied by the IIDP, rarely available in sufficient quantities) or βTC3 mouse insulinoma cells were used as positive controls for staining. Cells were stained with the primary antibodies listed in Table 3. All antibodies except for the rabbit anti-glucagon were pre-conjugated to fluorochromes to minimize background staining. Isotype controls were produced for all cell types in all staining procedures. Data were acquired on a BD LSR II instrument and analyzed with FlowJo Software (Ashland, Oreg.). Details on each staining procedure and type are elaborated below.

TABLE 3
Primary antibodies used for flow cytometric analysis of differentiating cells

| | | | | | | Permeabilization |
|---|---|---|---|---|---|---|
| Antigen | Host | Dilution | Manufacturer | Cat. # | Fluorochrome | Method |
| CXCR4 | Rat | 1:20 | BD Biosciences | 551510 | PE | N/A |
| PDGFRα | Rabbit | 1:10 | Santa Cruz | sc-338 | PE | N/A |
| | | | Biotechnology | | | |
| Sox17 | Goat | 1:10 | R&D Systems | IC1924A | APC | Saponin |
| Pdxl | Mouse | 1:20 | BD Biosciences | 562161 | PE | Methanol |
| Nkx6.1 | Mouse | 1:20 | BD Biosciences | 563338 | Alexa Flour | Methanol |
| | | | | | 647 | |
| Insulin | Rabbit | 1:50 | Cell Signaling | 8508S | PE | Saponin |
| | | | Technology | | | |
| Glucagon | Rabbit | 1:200 | LS Biosciences | LS-166525 | unconjugated | Saponin |

### Definitive Endoderm Marker Expression Analysis

For assessment of DE differentiation efficiency, cells from D5 of differentiation (end of stage 1) were incubated at room temperature for 2-5 minutes with TrypLE Express (Invitrogen, Grand Island, N.Y.), dissociated into a single cell suspension, filtered through a 70 μm mesh and washed with 1×PBS (Invitrogen, Grand Island, N.Y.) before being distributed into FACS tubes. After extracellular staining with antibodies against CXCR4 or PDGFR-α for 15 minutes in the dark at room temperature, the cells were washed and then permeabilized via saponin using the BD Cytofix/Cytoperm Kit (Catalog Number: 554714, BD Biosciences, San Jose, Calif.). The cells were incubated with anti-Sox17 for 30 minutes in the dark at room temperature before being washed and resuspended in 1×PBS for flow cytometric analysis.

### Pancreatic Transcription Factor Expression Analysis

For assessment of expression of pancreatic transcription factors (Pdx1, Nkx6.1 and NeuroD1), matrigel-seeded differentiating cell clusters on differentiation D15 (end of stage 4) were recovered by treatment with Dispase (Catalog Number: 354235, BD Biosciences, San Jose, Calif.) for 5 minutes at 37° C., followed by gentle suspension to further break down the matrigel. After washing with 1×PBS and centrifugation, the cell clusters were incubated with TrypLE Express for 5-10 minutes at room temperature. Following gentle resuspension and centrifugation, the cells were permeabilized using methanol as described below. The cells were fixed in 2% paraformaldehyde in PBS for 10 minutes at 37° C., followed by centrifugation, and resuspension of the vortexed cells in 1 mL of chilled Perm Buffer III (Catalog Number: 558050, BD Biosciences, San Jose, Calif.). The cells were incubated for 30 minutes on ice in sealed tubes. Subsequently, the cells were washed thrice in 3 mL Staining Buffer (1% FBS, 0.09% sodium azide in PBS) and finally resuspended in an appropriate volume of Staining Buffer that would allow for distribution of 100 μL of cell suspension to each FACS tube. The cells were incubated with fluorochrome-conjugated antibodies at the dilutions listed in Table 3 for 60 minutes at room temperature while protected from light. After one wash with 3 mL Staining Buffer, the cells were resuspended in 1×PBS for flow cytometric analysis.

### Pancreatic Hormone Expression Analysis

For assessment of expression of pancreatic hormones insulin and glucagon, matrigel-seeded differentiating cell clusters on differentiation D27 (end of stage 5) were recovered by treatment with Dispase (Catalog Number: 354235, BD Biosciences, San Jose, Calif.) for 5 minutes at 37° C., followed by gentle suspension to further break down the matrigel. After washing with 1×PBS and centrifugation, the cell clusters were incubated with TrypLE Express for 5-10 minutes at room temperature. Following gentle resuspension and centrifugation, the cells were permeabilized via saponin using the BD Cytofix/Cytoperm Kit (Catalog Number: 554714, BD Biosciences, San Jose, Calif.). For insulin staining, the cells were incubated with anti-inulin-PE for 30 minutes in the dark at room temperature before being washed and resuspended in 1×PBS for flow cytometric analysis. For glucagon staining, the cells were incubated with purified rabbit anti-human glucagon for 30 minutes in the dark at room temperature before being washed and incubated with anti-rabbit APC (1:100) for an additional 30 minutes in the dark at room temperature. Subsequently, the cells were washed and resuspended in 1×PBS for flow cytometric analysis.

### Dot Blot for 5-Methylcytosine

To detect the effectiveness of demethylation treatment, dot blots were performed on genomic DNA (gDNA) samples isolated from demethylated and nondemethylated control iPS cells to determine levels of 5-methylcytosine (5-MC). gDNA was isolated using the DNeasy Blood & Tissue Kit (Qiagen, Valencia, Calif.). First, Amersham Hybond-N+ (Catalog Number: RPN119B, GE Healthcare, Pittsburgh, Pa.), which is a positively charged nylon membrane, was placed on the surface of ultrapure water for at least 10 minutes to allow moistening of the membrane. In the meantime, 100 ng of DNA from each sample was distributed into separate Eppendorf tubes and the volume was equalized across all tubes by adding ultrapure water. Subsequently, 0.1 volume of 4 M NaOH (10× stock) and 0.1 volume of 100 mM EDTA at a pH of 8.2 (10× stock) were added to each sample to give a final concentration of 0.4 M NaOH and 10 mM EDTA. The mixture was vortexed and spun down. The DNA was then denatured at 99° C. for 7 minutes, chilled on ice, spun down and neutralized with 0.1 volume of 6.6 M ammonium acetate (10× stock) to give a final concentration of 0.66 M ammonium acetate. In the meantime, the membrane was removed from the water and allowed to dry on a pipette reload rack (with holes facilitating uniform dotting of DNA samples) until barely moist. This is ideal for allowing absorption of the DNA mixture into the membrane without being too dry. The DNA mixture was then spotted onto the membrane and air-dried for 30 minutes before being subjected to UV cross-linking (2× ‘auto cross-link’ on a Stratalinker). The antibody selected for the experiments was the monoclonal antibody of clone 33D3 (Catalog Number: A-1014-050, Epigentek, Farmingdale, N.Y.). The membrane was incubated overnight at 4° C. with the 5-mC antibody diluted at a concentration of 1:250 (4 μg/ml) in blocking solution (PBS containing 10% milk, 1% BSA, 0.1% Tween). After washing the blot 3 times with 0.1% Tween in PBS for 10 minutes each, the blot was subsequently incubated with HRP-conjugated anti-mouse antibody, diluted 1:2,000 in blocking solution for 1 hour at room temperature. The blot was then washed 3 times with 0.1% Tween in PBS at 10 minutes intervals. Finally, HRP signal was detected with a 5 minute incubation at room temperature in Amersham ECL Prime solution (Catalog Number: RPN2232, GE Healthcare, Pittsburgh, Pa.) and processed via X-ray films.

### Human Islets

Human islets used in this study were provided by the Integrated Islet Distribution program (IIDP). All methods and practices regarding the culture of islets were followed based on IIDP standard operating procedures. Briefly, immediately upon their receipt, islets were removed from the original flask and deposited into low attachment T75 flasks in the media they were supplied in. The islets were cultured upright for 48-72 hours in a 5% CO2 humid atmosphere at 37° C. to allow for the restoration of homeostatic metabolism prior to using the islets for any experimental procedures.

### Immunofluorescence and Confocal Microscopy

IPC clusters were cytospun onto SuperFrost Plus charged slides, rehydrated with 1×PBS, permeabilized with 0.2% Triton-X-100 and simultaneously blocked with PBS containing 10% BSA and 5% serum from the same species as the secondary antibody. After washing with PBS, slides were incubated at 4° C. overnight in primary antibody solutions or PBS (for the isotype control). The antibodies used for staining are detailed in Table 4. The slides were washed and then incubated with secondary antibodies for 1 hour at room temperature. Slides were mounted with VectaSheild Mounting Medium containing DAPI (Catalog Number: H-1200, Vector Laboratories, Burlingame, Calif.), covered with a coverslip, and sealed with nail polish. All experiments consisted of an appropriate negative control (undifferentiated iPS cells) and positive control (human islets). Each sample was stained in conjunction with an isotype control not exposed to the primary antibodies. For excised tissue obtained post-transplantation, after overnight fixation in 4% PFA, the tissue was embedded in paraffin for H&E staining or cryo-embedded in OCT using the Gentle Jane system for immunofluorescence analysis. For H&E staining, the tissue was sectioned, placed on SuperFrost Plus slides, and processed in an automatic H&E processor using a standard protocol. Immunofluorescent staining was performed after rehydrating slides in PBS and incubating the sections overnight at 4° C. After washing with PBS, slides were exposed to secondary antibodies for 1 hour at room temperature. Staining was documented by confocal microscopy using the Zeiss 710 Confocal Microscope at the Central Microscopy Research Facility at The University of Iowa. The list of antibodies used for immunofluorescence staining is provided in Table 4.

TABLE 4
Antibodies used for immunofluorescence staining

| PRIMARY ANTIBODIES | | | | | SECONDARY ANTIBODIES | | | | |
|---|---|---|---|---|---|---|---|---|---|
| Antigen | Host | Dilution | Manufacturer | Cat. # | 2° Antibody | | Dilution | Manufacturer | Cat. # |
| Insulin | Mouse | 1:100 | abcam | ab9569 | Goat α- | AF-568 | 1:200 | Life | A11019 |
| | | | | | Mouse | | | Technologies | |
| | | | | | Donkey | AF-568 | 1:200 | Life | A11057 |
| | | | | | α-Goat | | | Technologies | |
| Glucagon | Rabbit | 1:10 | LS Biosciences | LS- | Goat α- | AF-488 | 1:200 | Life | A11070 |
| | | | | C166525 | Rabbit | | | Technologies | |
| C-peptide | Rabbit | 1:100 | Cell Signaling | 4593S | Goat α- | AF-488 | 1:200 | Life | A11070 |
| | | | Technology | | Rabbit | | | Technologies | |
| Nkx6.1 | Mouse | 1:50 | DSHB | F55A10 | Goat α- | AF-568 | 1:200 | Life | A11019 |
| | | | | concentrate | Mouse | | | Technologies | |
| Insulin | Guinea | 1:50 | abcam | ab7842 | Goat α- | AF-488 | 1:200 | Life | A-11073 |
| (IHC-F) | Pig | | | | Guinea | | | Technologies | |
| | | | | | Pig | | | | |
| Somatostatin | Rat | 1:50 | abcam | ab30788 | Goat α- | AF-568 | 1:200 | Life | A-11077 |
| | | | | | Rat | | | Technologies | |

Quantitative-Real Time PCR (qRT-PCR)

The RNeasy Mini Kit (Qiagen, Valencia, Calif.) was used to extract total RNA and the SuperScript III First Strand Synthesis System for RT-PCR (Invitrogen, Grand Island, N.Y.) was used for reverse-transcription (Applied Biosystems, Foster City, Calif.) of 1 μg total RNA according to the manufacturer's instructions. cDNA (12.5 ng) was amplified by PCR using the SYBR Green PCR Master Mix (Applied Biosystems, Grand Island, N.Y.) in a 7500 Real-Time PCR System (Applied Biosystems, Grand Island, N.Y.). Data were normalized to undifferentiated human iPS cells using the ΔΔCt method, with TATA binding protein selected as the normalizer across samples. TBP was used after screening among three housekeeping genes, and it showed the most optimal amplification values relative to the other primers used in these experiments. Primers used for these experiments are listed in Table 5.

TABLE 5
List of primers used for quantitative RT-PCR

| | | | Tm | Tm |
|---|---|---|---|---|
| mRNA | Forward (5′→3′) | Reverse (5′→3′) | Forward | Reverse |
| TBP | TGTGCACAGGAGCCA | ATTTTCTTGCTGC | 59.1 | 55.9 |
| | AGAGT (SEQ ID NO: 1) | CAGTCTGG (SEQ | | |
| | | ID NO: 2) | | |
| IPF-1/PDX-1 | CCTTTCCCATGGATG | CGTCCGCTTGTTC | 53.1 | 55.7 |
| | AAG (SEQ ID NO: 3) | TCCTC (SEQ ID | | |
| | | NO: 4) | | |
| Glucagon | AAGCATTTACTTTGT | TGATCTGGATTTC | 55.6 | 57.2 |
| | GGCTGGATT (SEQ ID | TCCTCTGTGTCT | | |
| | NO: 5) | (SEQ ID NO: 6) | | |
| Glucokinase | TGCAGATGCTGGACG | GAACTCTGCCAG | 57.6 | 58.1 |
| | ACAG (SEQ ID NO: 7) | GATCTGCTCTA | | |
| | | (SEQ ID NO: 8) | | |
| Ghrelin | TGAACACCAGAGAG | GCTTGGCTGGTGG | 58.1 | 58.6 |
| | TCCAGCA (SEQ ID | CTTCTT (SEQ ID | | |
| | NO: 9) | NO: 10) | | |
| Somatostatin | CCCCAGACTCCGTCA | TCCGTCTGGTTGG | 57.5 | 57.1 |
| | GTTTC (SEQ ID NO: 11) | GTTCAG (SEQ ID | | |
| | | NO: 12) | | |
| Insulin | AAGAGGCCATCAAG | CAGGAGGCGCAT | 56.4 | 58.1 |
| | CAGATCA (SEQ ID | CCACA (SEQ ID | | |
| | NO: 13) | NO: 14) | | |

### Dithizone Staining

Freshly prepared dithizone solution was used for all experiments. First, 20 mg of dithizone (Catalog Number: D5130, Sigma-Aldrich, St. Louis, Mo.) was added to 0.6 mL of 95% ethanol in a 15 mL conical tube. Subsequently, 1-5 drops of ammonium hydroxide was added and the resulting orange stock mixture was vortexed thoroughly until completely dissolved. 0.3 mL of this stock solution was dispensed in 99.7 mL of 1×PBS and the pH was adjusted to 7.4 with 1N HCl. Islets (supplied by the IIDP) or picked IPC clusters were added to separate wells each containing 200 μL of the final dithizone solution in a 96 well plate. The cell clusters were incubated for 2-5 minutes before images were captured using a standard light microscope (Nikon Eclipse, TS100) attached to a color camera.

### Transmission Electron Microscopy

IPC clusters were dissociated from matrigel via gentle suspension of the matrigel scaffold and washed with PBS. Dispase was not utilized due to the risk that the enzyme might compromise the integrity of cellular structures. Meanwhile, human islets (supplied by the IIDP) were isolated and pelleted after washing with PBS. Cell cluster pellets were incubated overnight in 2.5% gluteraldehyde in 0.1 M Sodium Cacodylate buffer at 4° C., although samples are generally considered to be indefinitely stable in this buffer. After rinsing in 0.1 M phosphate buffer twice (4 minute incubations each), the clusters were fixed using the secondary-staining, lipid-fixing agents 1% OsO₄/1.5% Potassium Ferrocyanide in 0.1 M phosphate buffer for 30 minutes on a shaker platform. The clusters appeared black at this point and were rinsed twice in double distilled H₂O (ddH₂O). Subsequently, the clusters were incubated in ultrasaturated 2.5% Uranyl Acetate stock solution for 5 minutes. The cell clusters were then successively washed in higher concentrations of ethanol in order to dehydrate the samples. Finally, the clusters were embedded in Spurr's resin and placed in Beem Capsules in a 70° C. oven overnight. After microtomy and depositing the embedded sections onto copper grids, the grids were stained facedown in Uranyl Acetate droplets for 3 minutes. Subsequently the grids were rinsed in water, dried, and stained for 2 minutes in Lead Citrate droplets. NaOH pellets in the petri dishes with the Lead Citrate droplets were used to trap air and prevent oxidation of the Lead Citrate buffer. After rinsing and drying, the grids were replaced into grid holders and imaged using the JEOL JEM 1230 Transmission Electron Microscope. This microscope is located in the Central Microscopy Research Facility at the University of Iowa and was operated by Dr. Chantal Allamargot of the core facility.

### Mice and Transplantation

Immunodeficient Rag2^{-/-}γc^{-/-} mice (B6 background) of 6-10 weeks of age were purchased from Taconic Farms and used for all animal experiments. All animal procedures were approved by the Institutional Animal Care and Use Committee (IACUC) at the University of Iowa and the Iowa City VA Medical Center, and procedures were conducted in accordance with NIH guidelines.

The multiple low dose (MLD) protocol was used for induction of hyperglycemia using Streptozotocin. For induction of diabetes, pre-weighed mice were placed on a 4 to 6 hour fast by placing the mice in a fresh cage with a new food rack that does not have food. 3.5 hours into the fast, fresh Sodium Citrate buffer was prepared by weighing out 0.735 g of enzyme grade Sodium Citrate (Catalog Number: S279-500, Fisher Scientific, Waltham, Mass.) and dissolving it in 25 mL of ddH₂O. The pH was adjusted to 4.5 using HCl and the buffer placed on ice. Subsequently, a sufficient amount of powdered Streptozotocin (STZ, Catalog Number: 572201, Millipore, Billerica, Mass.) was placed into a 1.5 ml Eppendorf tube protected from light with aluminum foil so that each mouse would receive 100 mg STZ/kg mouse body weight. At 4 hours post-fast, STZ was resuspended in fresh Sodium Citrate buffer and injected i.p. within 5 minutes of dissolution so that each mouse received 100 μL of STZ solution to achieve the dose of 100 mg/kg. After commencing with the injections, food and water were supplied to all of the mice. This procedure was repeated 2 days after the first dose to achieve a dose of 50 mg/kg. The blood glucose levels were determined 2 days later, and any mice that were not hyperglycemic (>300 mg/dL) received a third dose of 50 mg/kg STZ. After a maximum of three doses of STZ, blood glucose levels and weights of the STZ-injected mice were measured, and only mice showing evidence of hyperglycemia (>300 mg/dL blood glucose) were used for transplantation experiments. Hyperglycemic mice (>300 mg/dl blood glucose) were anesthetized and injected with IPCs s.c. into the right shoulder flank, which was shaved and marked to indicate transplant site. Mice received 500 IEQs of islets or 900 IPC clusters. After 2 weeks of transplantation, mice were weighted and their blood glucose levels measured every 7 days.

For the glucose tolerance test, pre-fasting blood glucose levels were recorded and the mice were then fasted for 16 hours in new cages with only water to prevent access to food remnants. Weights of the mice were determined to ensure accurate dosage of glucose. Following the fast, blood glucose levels were again determined (0 min), and the mice were injected i.p. with 2 mg/kg of (D)-+-Glucose solution suspended in water. Blood glucose levels were assessed at 15, 30, 60, 90, 120, and 240 minutes after glucose challenge.

### Statistical Analysis

Evaluation of experimental data for significant differences was performed through the Students t test, which was conducted using the Prism software package (GraphPad Software). p<0.05 was considered significant for these studies. Unless noted otherwise, all experiments were repeated at least 3 times.

### Results

Differentiation of T1D and Non-Diabetic (ND) iPS Cells into Definitive Endodermal (DE) Cells

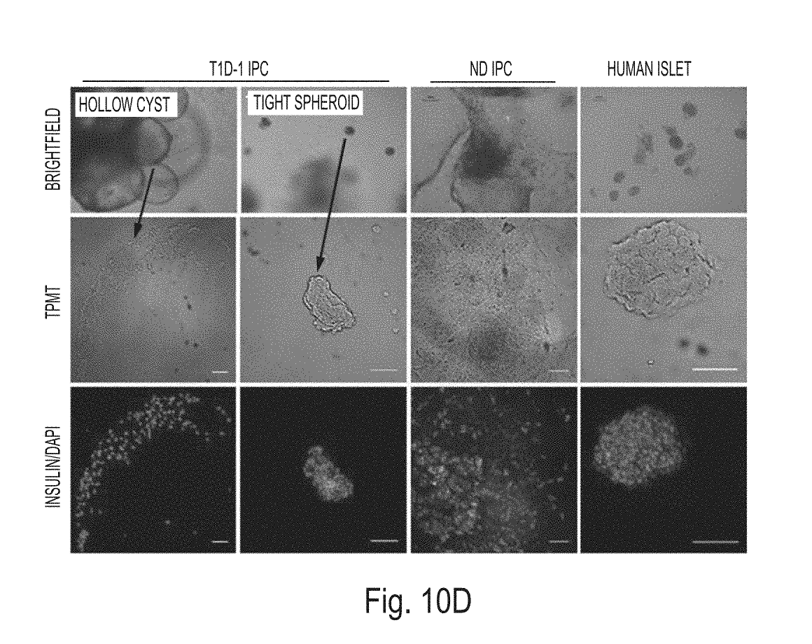

Data has been published on the differentiation of iPS cells from healthy individuals (Raikwar et al., 2015; D'Amour et al., 2006; Kroon et al., 2008; Rezania et al., 2012; Xie et al., 2013; Zhang et al., 2009; Pagliuca et al., 2014; Rezania et al., 2014). Here, additional critical signaling cues that instruct iPS cells to become pancreatic β-cells were incorporated in order to further improve the yield of IPCs (FIG. 10A). The differentiation process drives the cells through five stages lasting a total of 27 days. The first stage of differentiation towards IPCs is the conversion of iPS cells into definitive endodermal cells (DE cells) (Spence et al., 2007) through Activin A and Wnt3a signaling¹⁰ (Rezania et al., 2012) (Stage 1). DE cells are characterized by the expression of CXCR4 and Sox17. The resulting cells are next exposed to FGF-7, which acts to convert the DE cells into the posterior foregut (Rezania et al., 2012) (Stage 2). Following this step, the cells are treated with retinoic acid and SANT-1, an inhibitor of the Shh pathway (Rezania et al., 2012), in order to posteriorize the gut tube and promote pancreatic differentiation as opposed to hepatic specification (Mfopou et al., 2010). Reinforcing this, Noggin is added to this media cocktail as well¹⁰, and it functions to inhibit BMP signaling (Groppe et al., 2002). The aim of this phase is to generate Pdx1⁺ pancreatic precursor cells (Stage 3). The cells are then instructed to become pancreatic endocrine precursor cells by carrying over certain cues from Stage 3 and supplementing them with inhibitors of TGF-β signaling (Loh et al., 2012) and suppression of Notch signaling (Pagliuca et al., 2014; Rezania et al., 2014), which actively inhibits endocrine differentiation by the process of lateral inhibition (Murtaugh et al., 2003). Suppression of TGF-β signaling is accomplished by the treatment with ALK5 inhibitor II (Gellibert et al., 2004). Repression of Notch signaling is indirectly accomplished by blockade of its ligand, γ-secretase, using DAPT (Firth et al., 2014). In addition, this stage of differentiation introduces several drivers of pancreatic β-cell development, such as thyroid hormone (T3) (Pagliuca et al., 2014; Rezania et al., 2014) and the incretin GLP-1 (Thatava et al., 2011) (Stage 4). Finally, in the last stage of differentiation, specific inducers of insulin are added in addition to agents that promote the maturation and development of pancreatic β-cells, such as nicotinamide (Otonkoski et al., 1993; Shahjalal et al., 2014; Ye et al., 2006), IGF-1 (Thatava et al., 2011; Withers et al., 1999), GLP-1 and T3 (Stage 5).

Using this protocol, ND and T1D iPS cells were first differentiated into DE cells in parallel and the efficacy of differentiation was assessed on day 5 by determining the expression of CXCR4, Sox17, and PDGFR-α. Co-expression of CXCR4 and Sox17 typifies lineage commitment to the endoderm. Undifferentiated iPS cells were utilized as negative controls (FIG. 10B). Both T1D and ND differentiated cultures contained >90% CXCR4⁺ Sox17⁺ endodermal cells (FIG. 10B). Additionally, these cells were mostly PDGFR-α⁺ (FIG. 10B), which suggests that they are true endodermal cells and not arrested in the transitory, immature mesendodermal state (Tada et al., 2005). Thus, we concluded that we were able to achieve a near pure population of DE cells from both ND and T1D iPS cells at comparable yields. For further differentiation into IPCs, the 2D differentiating DE cells were dissociated by scraping the monolayers and depositing these cell clusters into matrigel blocks. The cells coalesced into discrete spheroids that embedded into the matrigel within 24 hours, allowing differentiation of the cells on a 3D platform.

T1D iPS Cells Predominantly Derive Hollow Cysts that do not Express Insulin

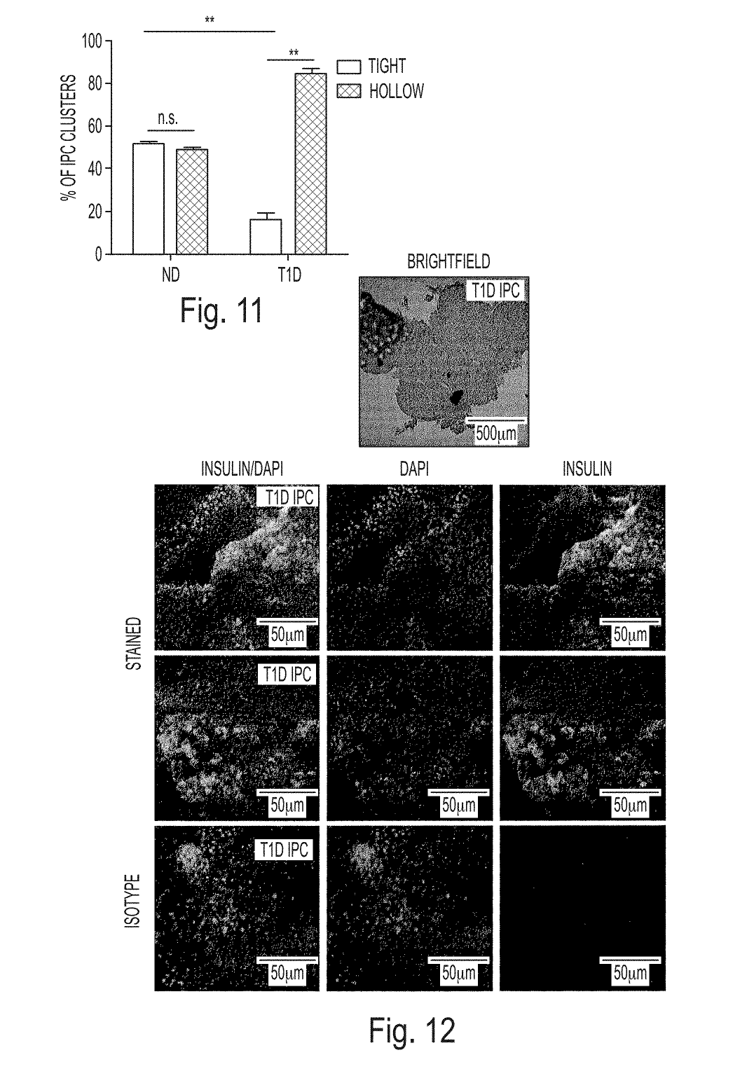

Early in the 3D differentiation procedure, we recognized that the DE cells from both T1D and ND cultures coalesced into compact cell clusters. However, in the final stage of the differentiation, which lasts 10 days, the formation of clusters with two distinct morphological phenotypes was observed: hollow cysts that appeared to be like bubbles, and compact spheroids (FIG. 10C). Strikingly, the T1D cultures consisted almost entirely of hollow cysts, whereas the ND iPS cells gave rise to a ratio of 50:50 of hollow cysts to compact spheroids (FIG. 11). The cysts emerged gradually with the differentiation process, becoming more prominent towards the end of the differentiation. To further characterize these hollow cysts and compact spheroids, the cells were stained for insulin. The hollow cysts collapsed upon fixation in paraformaldehyde (FIG. 10D), which is an observation that is consistent with what has been described in Greggio et al., 2013. When we stained these structures for insulin, the compact spheroids, but not the hollow cysts, stained positive for insulin (FIG. 10D). It is not clear whether these cysts constitute some precursor form of pancreatic cells.

Remarkably, the morphology of the compact spheroids resembled that of pancreatic islets (FIG. 10D). This result suggested that to optimize the yield of IPCs, the number of the compact spheroids needed to increase. In addition to the hollow cysts in the T1D cultures, we also rarely found large compact organoid-like structures that strongly expressed insulin, (FIG. 12).

### T1D iPS Cells Give Rise to Significantly Fewer Insulin-Expressing Cells

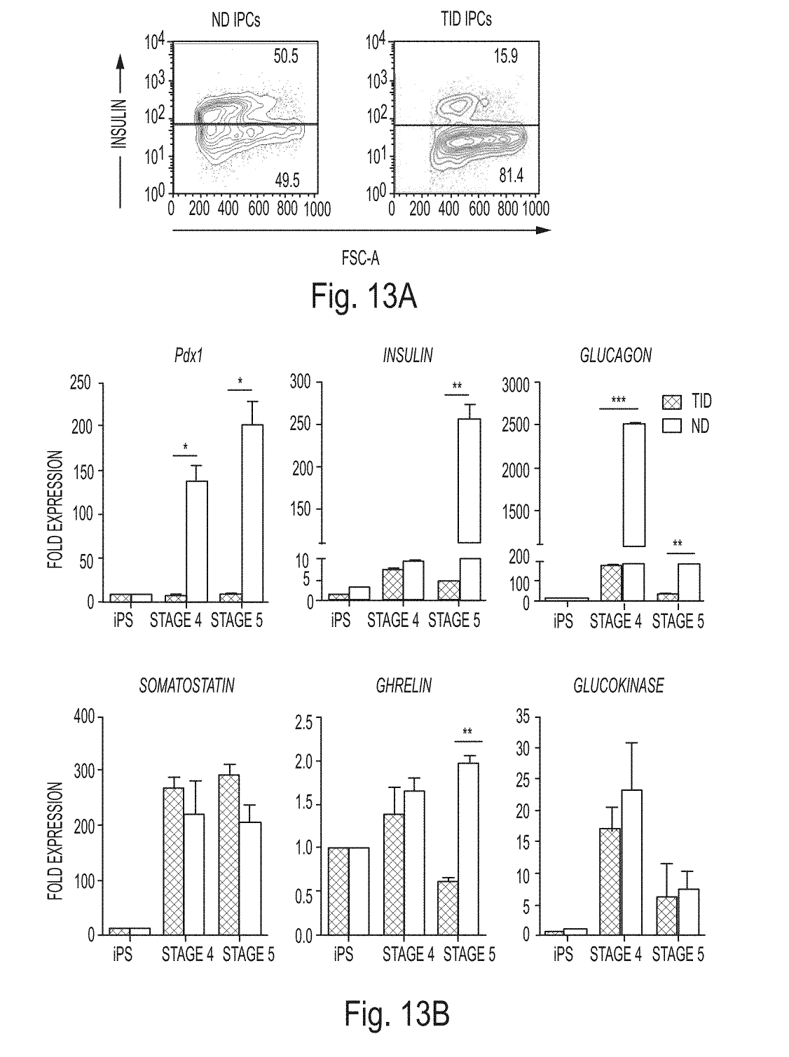

As determined by flow cytometry, T1D iPS cells poorly differentiated into IPCs (FIG. 13A). The yield of IPCs derived from T1D iPS cells was 15.9% compared to 50.5% in the ND iPS cells (FIG. 13A). The 50.5% yield of IPCs derived from ND iPS cells is comparable to the frequency of β-cells found in primary human islets³³. This yield of IPCs from ND iPS cells is far superior when compared to previous reports, where the yield of IPCs was only 10-15% (Raikwar et al., 2015; D'Amour et al., 2006; Kroon et al., 2008; Rezania et al., 2012; Xie et al., 2013; Zhang et al., 2009; Pagliuca et al., 2014; Rezania et al., 2014). It was concluded that 3D differentiation is superior for iPS cell differentiation compared to 2D differentiation. However, despite the effectiveness of our protocol with ND iPS cells, only 15.9% of the T1D culture expressed insulin. Thus, these data allow for the conclusion that the yield of insulin-expressing cells from T1D iPS cells is impaired.

### T1D iPS Cell-Derived Differentiating Cells Poorly Express Pdx1

To further investigate why the differentiation of T1D iPS cells was impaired, next the immunofluorescence and flow cytometry data was corroborated by gene expression studies. Gene expression for several genes was studied in Stages 4 and 5 of both T1D and ND differentiation cultures. As can be seen in the top panel of FIG. 13B, the expression of the Insulin transcript in the ND cells increases from Stage 4 to Stage 5, and this is accompanied by a decline in the expression of Glucagon, suggesting lineage commitment of the cells toward insulin-expressing cells in the last stage of the differentiation. However, in the T1D differentiating cultures, it was observed significantly lower expression of Insulin at both Stages 4 and 5, confirming previous results. Significantly poorer expression of Glucagon was observed in the T1D cultures compared to ND cultures. The expression of other genes, such as Somatostatin and Glucokinase was not significantly different between the T1D and ND cultures, and Ghrelin showed a very small but significant difference (FIG. 13B).

To determine if the inefficiency in differentiation manifests earlier than the last stage of differentiation, the expression of Pdx1 was determined in T1D and ND differentiating cultures. Pdx1 is the master regulator gene in the pancreas and its expression appears midway through the differentiation process (Rezania et al., 2012). In ND cultures, Pdx1 is expressed in high levels in Stage 4 (FIG. 13B), which precedes the expression of Insulin in Stage 5, consistent with embryonic development of the pancreas Parnaud et al., 2006). Pdx1 levels continue to increase in Stage 5 in the ND culture. However, at both Stages 4 and 5, the T1D cultures expressed significantly lower levels of Pdx1 than the ND culture (FIG. 13B). Pdx1 is indispensable for the development of pancreatic β-cells (Spence et al., 2007). Pdx1 knockout mice fail to form a pancreas (Stoffers et al., 1997), which is evidence for how critical this gene is. This likely explains why the expression of downstream genes, such as Insulin, is also impaired in the T1D differentiating cultures.

### Effective Differentiation Outcomes Requires Precise Temporal Modulation of Demethylation Treatment

Thus, the differentiation of T1D iPS into IPCs was impaired. Considering the importance of epigenetics in cell differentiation and the poor expression of Pdx1 in T1D differentiating cultures, it was hypothesized that epigenetic barriers were likely responsible for the poor yield of IPCs derived from T1D iPS cells. To address this problem, we utilized 5-aza-2′-deoxycytidine (5-aza-DC), a potent demethylating agent that inhibits the DNA methyltransferase (Dnmt) (Christman, 2002).

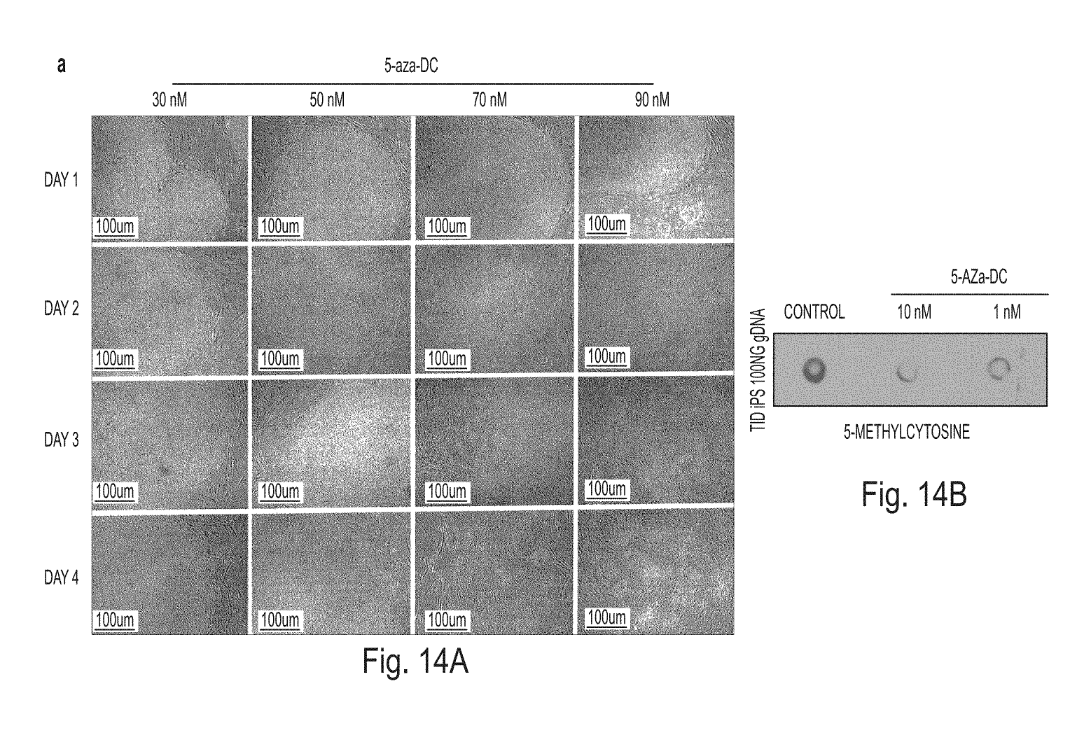

A dose-screen experiment was established to identify the optimal dose of 5-aza-DC for treatment that would preserve cell viability while effectively demethylating the DNA of the cells. In order to ensure that the integrity of the differentiating cells would be preserved, we utilized smaller doses of 1 nM and 10 nM to minimize cell toxicity (FIG. 14A). A dot blot assay (Pennarossa et al., 2013) for 5-methylcytosine on gDNA isolated from iPS cells that were treated with 5-aza-DC showed loss of methylation in these cells (FIG. 14B).

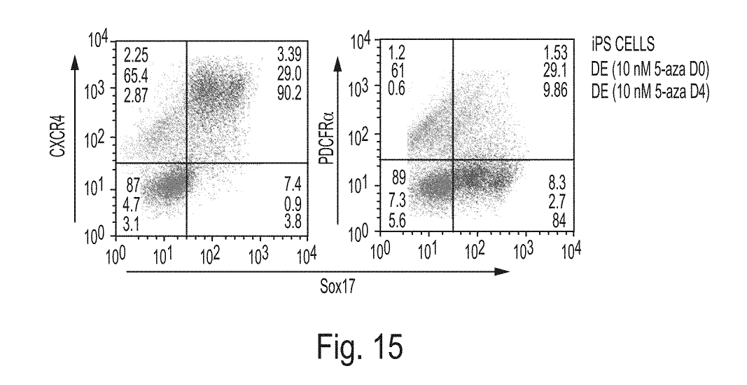

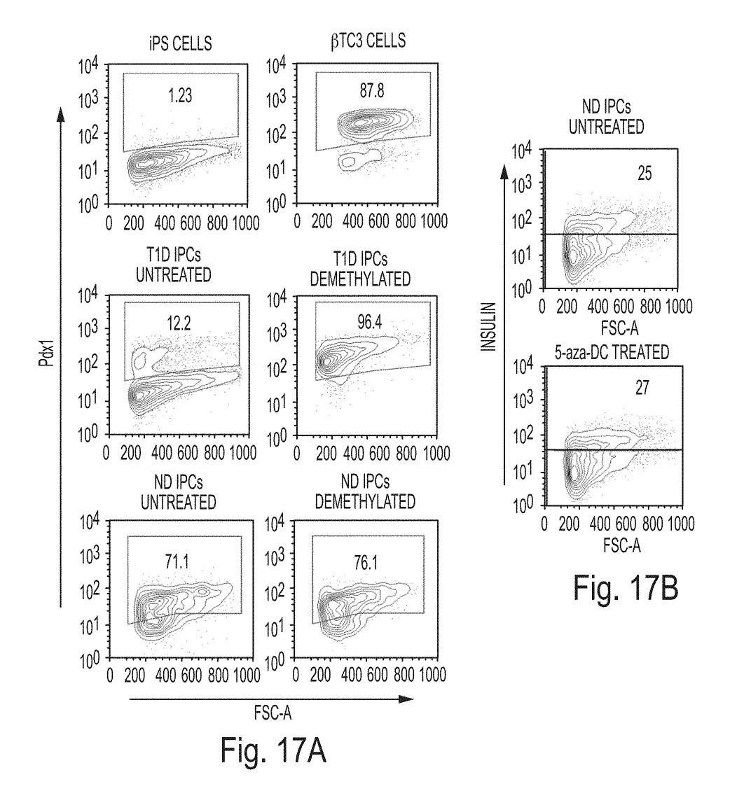

Two possible time points were considered for the demethylation treatment: 1) at the start of the differentiation into DE cells, or 2) after the generation of DE cells, before the cells progress into the stage in which Pdx1⁺ cells are generated. It was observed that the treatment of iPS cells with 10 nM 5-aza-DC on day 0 (before initiating the generation of DE cells) resulted in cells on day 5 that were arrested in the immature CXCR4⁺ PDGFRα⁺ Sox17⁻ mesendodermal state (FIG. 15). In contrast, demethylation of the cells on day 4 yielded a pure population of CXCR4⁺ Sox17⁺ PDGFRα⁻ DE cells, similar to what we were able to generate without any demethylation agent. This experiment led to the conclusion that the demethylation treatment was ideal after the generation of DE cells, a phase during which cell division is elevated.

### Demethylation of T1D DE Cells Rescues the Expression of Pdx1 and Leads to the Generation of Islet-Like Compact Cell Clusters

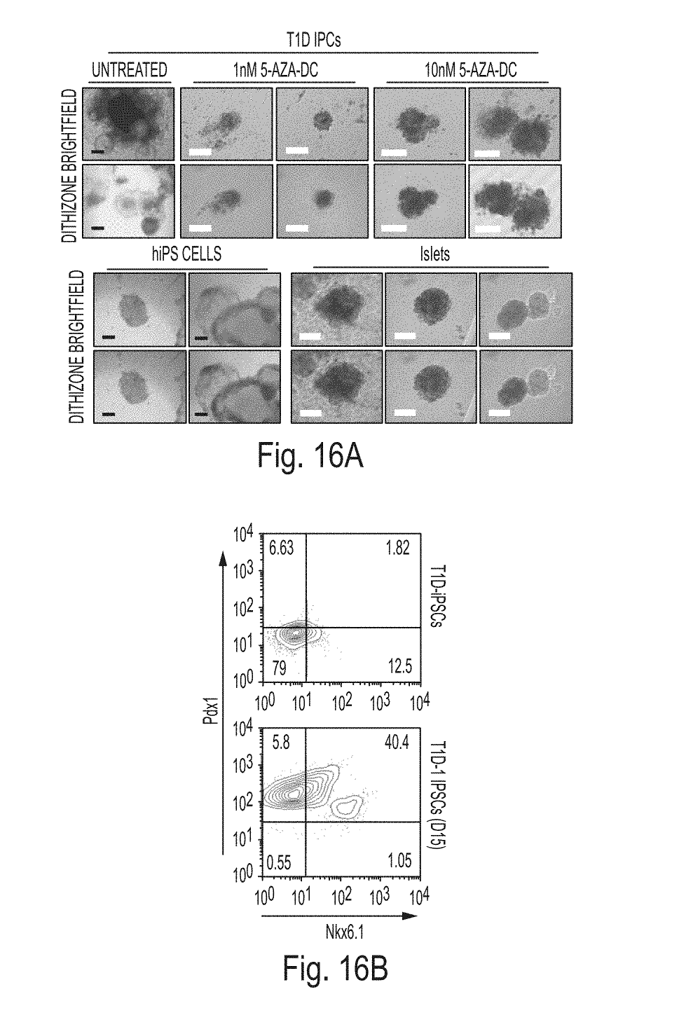

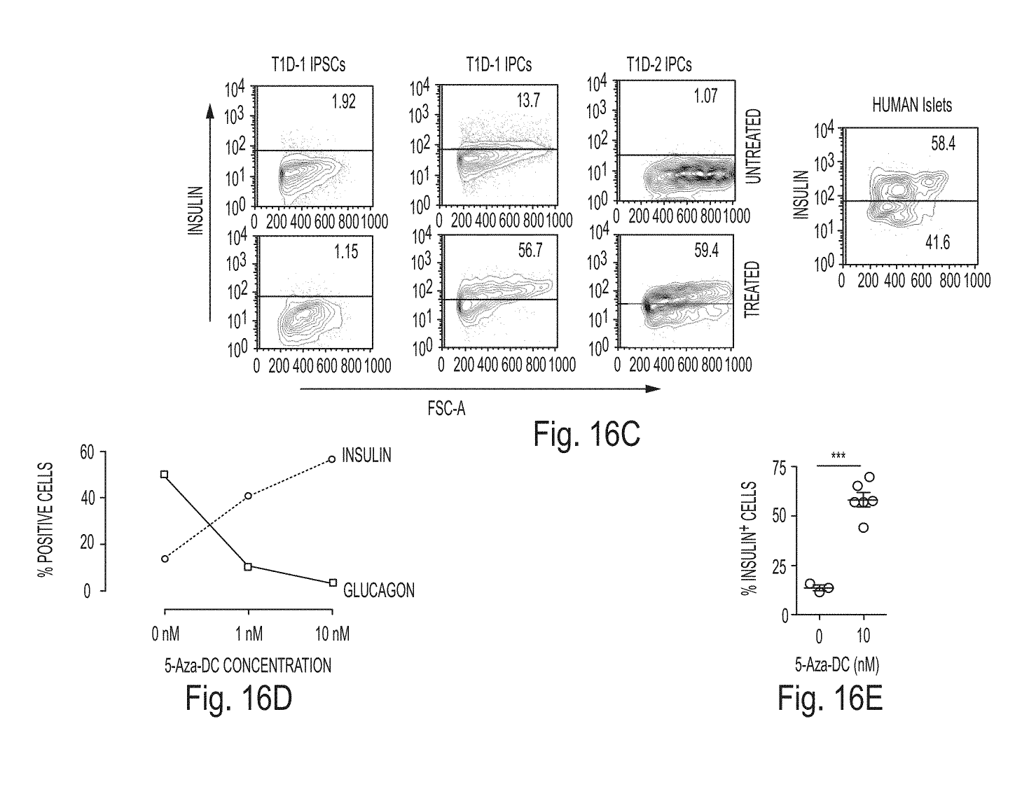

A full differentiation of T1D iPS cells into IPCs was initiated, demethylating the DE cells on day 4 for 18 hours before transferring them onto matrigel. A striking impact of the demethylation treatment in the T1D cultures was observed. Typically, T1D iPS cells gave rise to a disorganized mix of cysts and spheroids, with a dominant presence of hollow cysts. At both doses tested, 5-aza-DC treatment instead promoted the formation of compact cell clusters that uniquely resembled human islets (FIG. 16A). Next, these cell clusters were stained with dithizone, which when positive points to the presence of insulin Shiroi et al., 2002). Dithizone staining revealed the strong red color of the compact clusters found in the 5-aza-DC treated cultures, which was reminiscent of islets (FIG. 16A). This was in contrast to what was observed in the IPCs derived from untreated iPS cells, which stained brown in a manner similar to undifferentiated iPS cells (FIG. 16A).

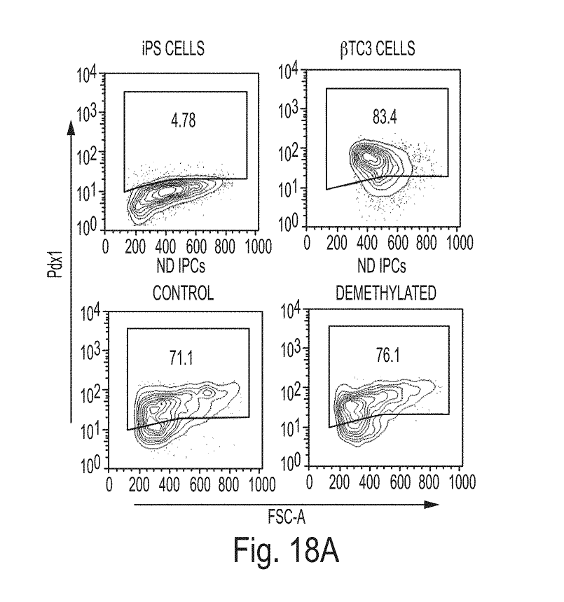

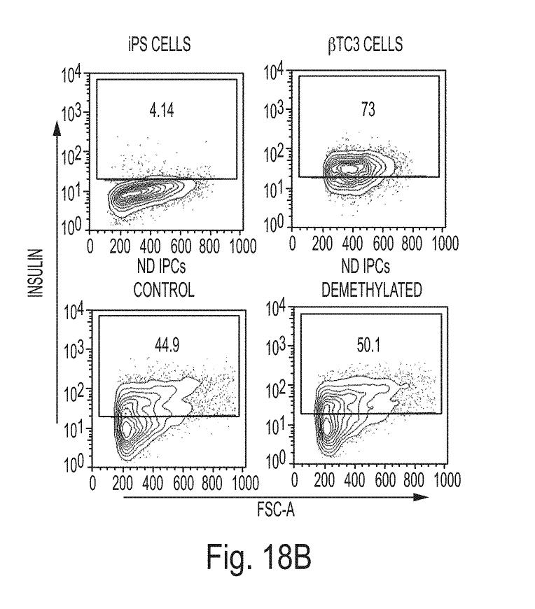

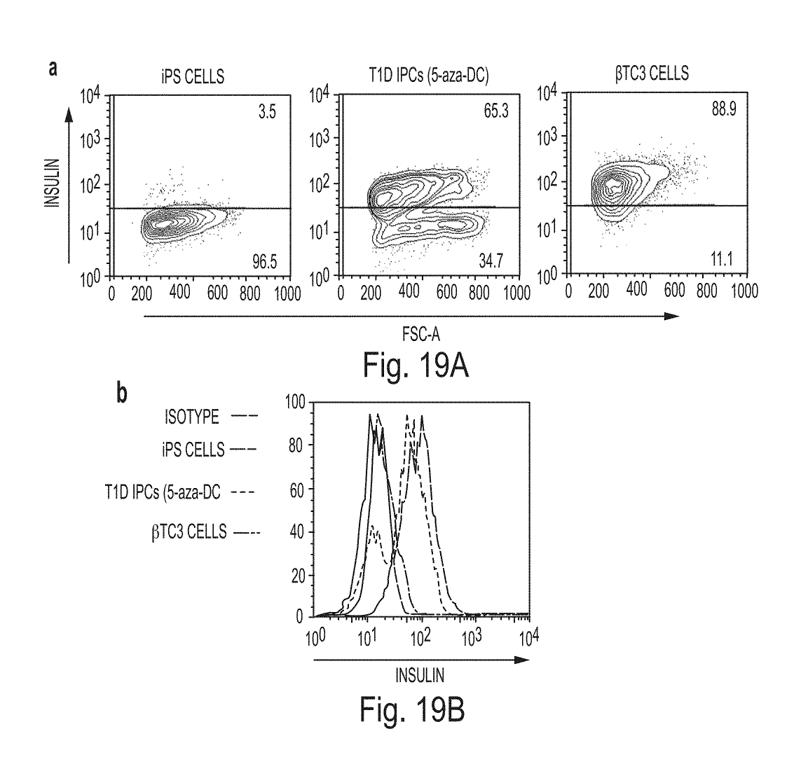

Next, it was assessed whether the demethylation rescued the expression of Pdx1 in the differentiating T1D cultures. As described above, without the demethylation step, the yield of insulin-expressing cells from T1D iPS cells was approximately 15% at the end of Stage 5, which is consistent with the 12% yield of Pdx1⁺ pancreatic progenitor cells observed in regular differentiations at the end of Stage 4 (FIG. 17). However, after demethylation, robust expression of Pdx1 (about 95%) was observed at the end of Stage 4 (FIG. 17). Demethylation of differentiating ND cells did not significantly improve the yield of Pdx1⁺ cells or insulin-expressing cells (FIG. 18). In T1D differentiating cultures, a significant proportion of cells co-expressed Pdx1 and the pancreatic β-cell specific transcription factor Nkx6.1, which is critical for maintaining the identity and function of pancreatic β-cells (Schaffer et al., 2013; Taylor et al., 2013) (FIG. 10B). Altogether, these data suggested that the transient demethylation treatment allowed the expression of Pdx1 in differentiating T1D cultures.

Demethylation of T1D DE Cells Significantly Improves the Differentiation of DE Cells into Pancreatic Cells

These data then led us to wonder if the demethylation treatment enhanced the expression of downstream targets of Pdx1, such as insulin (Murtaugh, 2007), and made the cells more receptive to differentiation cues while averting commitment towards alternative lineages. Specifically, it was sought to determine the proportion of IPCs relative to those that stain for glucagon. Most reports published so far generate multi-hormonal cultures that express both insulin and glucagon (Raikwar et al., 2015; D'Amour et al., 2006; Kroon et al., 2008; Rezania et al., 2012; Xie et al., 2013; Zhang et al., 2009; Pagliuca et al., 2014; Rezania et al., 2014)).

As can be seen in FIG. 16C, the proportion of glucagon-expressing cells was high when the iPS cells were not demethylated, whereas they were depleted after the demethylation step (second column in FIG. 16C). However, with demethylation at a dose of 10 nM 5-aza-DC, 56% of the cells expressed insulin, with a much smaller portion of the cells expressing glucagon (FIG. 16C, third and fourth columns). This result is summarized in FIG. 16D, which demonstrates that, at higher concentrations of the demethylating agent, nearly all the glucagon secreting cells are gone. Because the highest yield resulted from treatment of the cells with 10 nM 5-aza-DC, this dose was chosen for all subsequent experiments. This experiment has been repeated multiple times and as many as 65% insulin⁺ cells were measured in some cultures (FIG. 19). Pooling of data from several differentiations reveals that 5-aza-DC treatment consistently enhances the yield of IPCs (FIG. 16E).

T1D IPCs Derived from Demethylated DE Cells Express Pancreatic β Cell-Specific Markers and Possess Insulin Granules at Similar Levels to Those in Cadaveric β-Cells

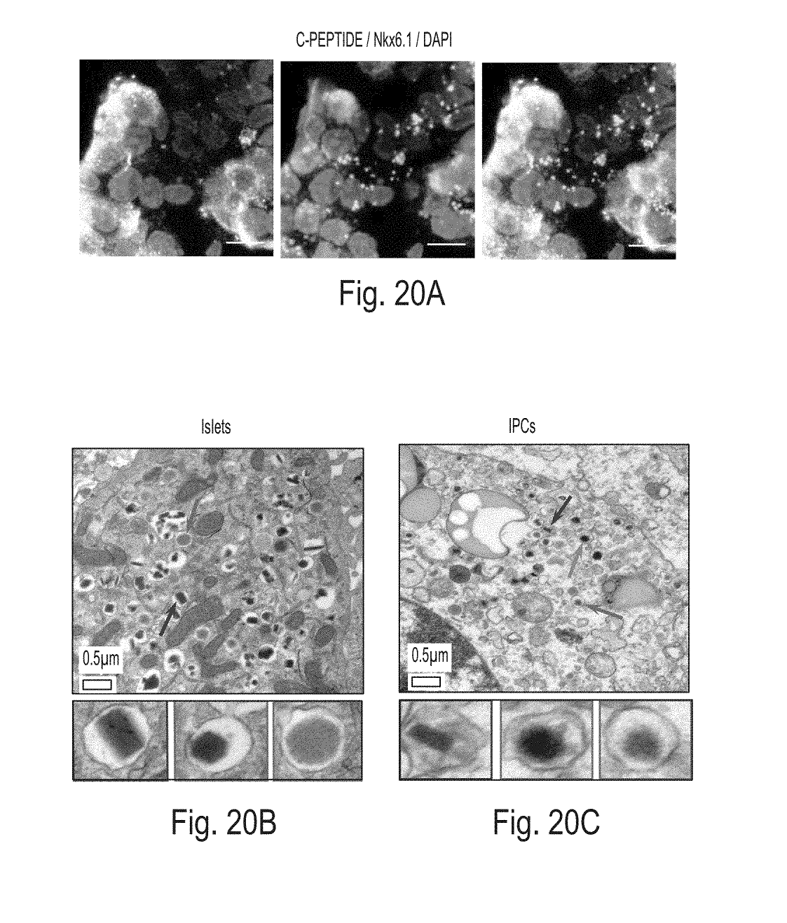

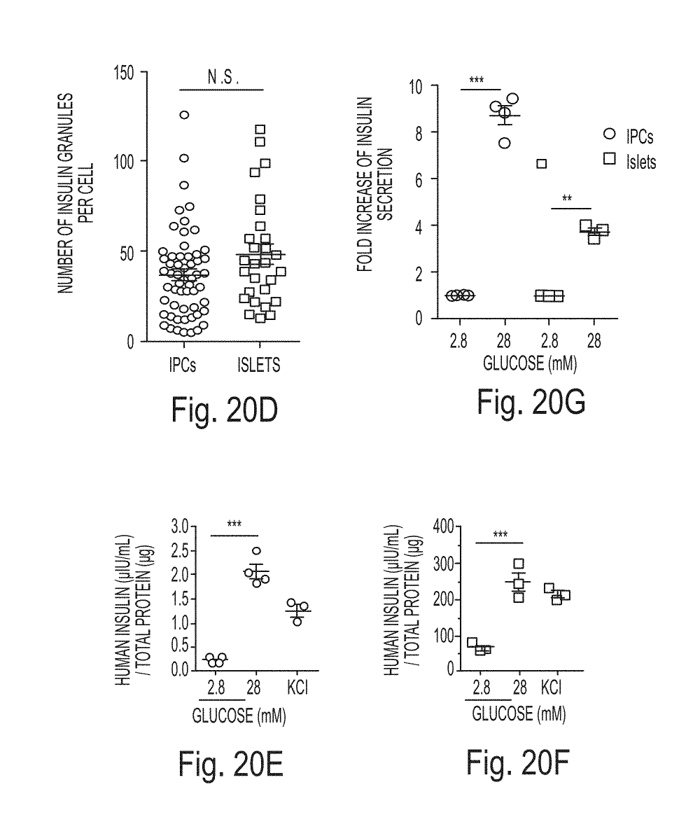

Published protocols for the generation of IPCs from ES cells generally give rise to a multi-hormonal pool of cells, of which very few cells express only insulin (Raikwar et al., 2015; D'Amour et al., 2006; Kroon et al., 2008; Rezania et al., 2012; Rezania et al., 2012; Xie et al., 2013; Zhang et al., 2009; Pagliuca et al., 2014; Rezania et al., 2014). The above described protocol allowed for the selective generation of insulin-expressing cells from T1D iPS cells while generating very few glucagon-expressing cells. Immunofluorescence analysis of the differentiated cell clusters showed that the cells were mostly insulin-secreting with a very small percentage of glucagon secreting cells (FIG. 20A). Since the rest of the cells are Pdx1 expressing cells, it was assumed they are all pancreatic and may be expressing other pancreatic hormones.

Additionally, staining for the nuclear transcription factor Nkx6.1, which is critical for the maintenance of pancreatic β-cell function and identity (Schaffer et al., 2013; Taylor et al., 2013), was conducted. The cells were co-stained for the insulin precursor C-peptide, which is synonymous with de novo production of insulin (Thatava et al., 2011; Chen et al., 2013). As evidenced in FIG. 20B, T1D IPCs derived via demethylation show robust expression of C-peptide in the cytoplasm as well as strong nuclear expression of Nkx6.1 (FIG. 20B).

Next, the ultrastructure of these cells was analyzed by Transmission Electron Microscopy. A unique pancreatic β-cell like morphology of the granules contained in the T1D IPCs (FIG. 20C, upper panel) was observed. Unlike the first protocol that was described in Example 1, these granules are identical to those present in cadaveric β-cells. Insulin granules undergo various stages of maturation that are differentiated by the shape and darkness of the core Pagliuca et al., 2014; Rezania et al., 2014). The most mature insulin granules are angular due to the hexamer complexation of insulin with zinc, which creates a crystalline shape Norris and Carr, 2013). However, insulin granules universally possess a characteristic “halo” that is not found on any other hormone granule (Norris and Carr, 2013). This feature is thus a unique and specific indication of β-cell like phenotype.

As can be seen in the lower panel of FIG. 20C, IPCs resemble pancreatic β-cells in their possession of the three different granule subtypes, all of which have the characteristic halo. Additionally, the number of granules found in the IPCs was not statistically different from the number of granules in primary human islets (FIG. 20D). Thus, a protocol was established for the generation of human IPCs from T1D iPS cells that strongly resemble human β-cells in their ultrastructure in addition to their expression of insulin and other pancreatic β-cell specific markers.

### T1D IPCs are Functional and are Glucose Responsive

Perhaps the most important criterion for defining the authenticity of the generated IPCs is to observe whether they secrete insulin when stimulated with high glucose (Yechoor and Chan, 2010). PSC-generated IPCs have failed to respond to glucose stimulation until very recently. Two recent reports described for the first time the generation of glucose-responsive cells from human ES cells (Pagliuca et al., 2014; Rezania et al., 2014). However as of yet, functional, glucose-responsive IPCs from human iPS cells of T1D patients have not been generated. This is the first report that may be more clinically relevant because self-tailored IPCs will be generated in T1D patients.

To address the glucose-responsiveness of these IPCs, T1D IPC cell clusters were subjected to a glucose stimulated insulin secretion (GSIS) assay. As evidenced in FIG. 20E, these IPCs are glucose-responsive in a manner that has never been observed before in IPCs derived from T1D iPS cells. Although the amount of insulin secreted by IPCs is significantly lower compared to that secreted by islets (FIG. 20F), the fold-increase in insulin production by IPCs is higher than that for islets (FIG. 20G). This result is a remarkable demonstration of the superiority of this protocol compared to prior reports in generating authentic, functional IPCs from human iPS cells derived from T1D patients.

### Rapid Correction of Hyperglycemia in Diabetic Mice by T1D IPCs

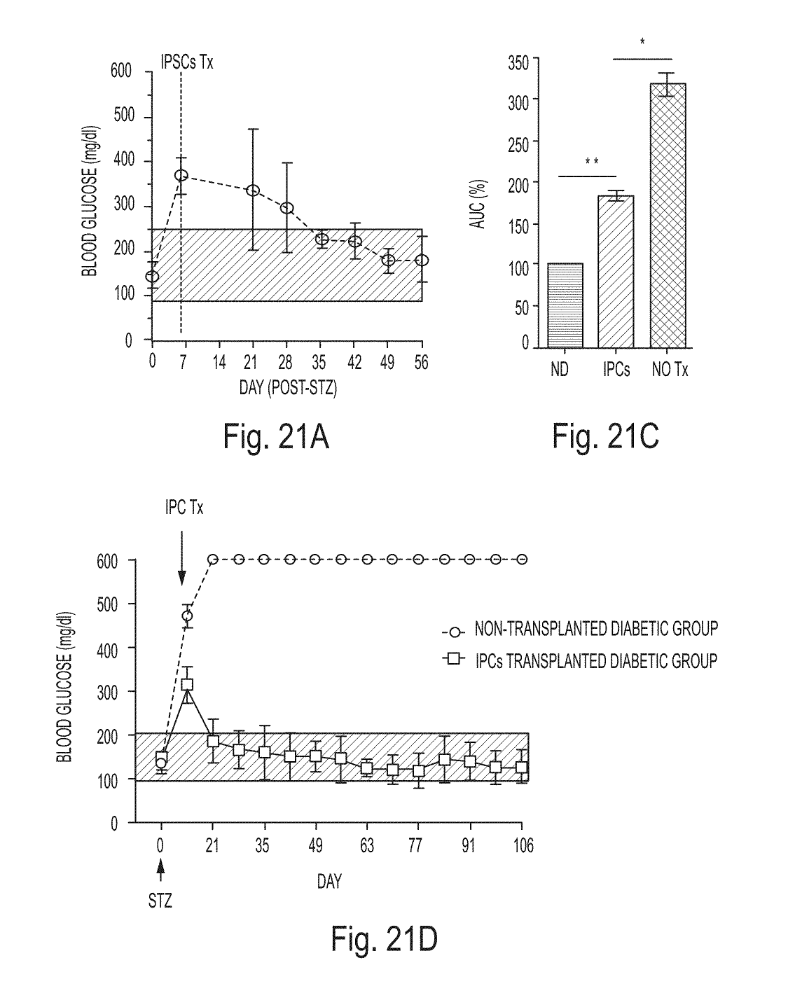

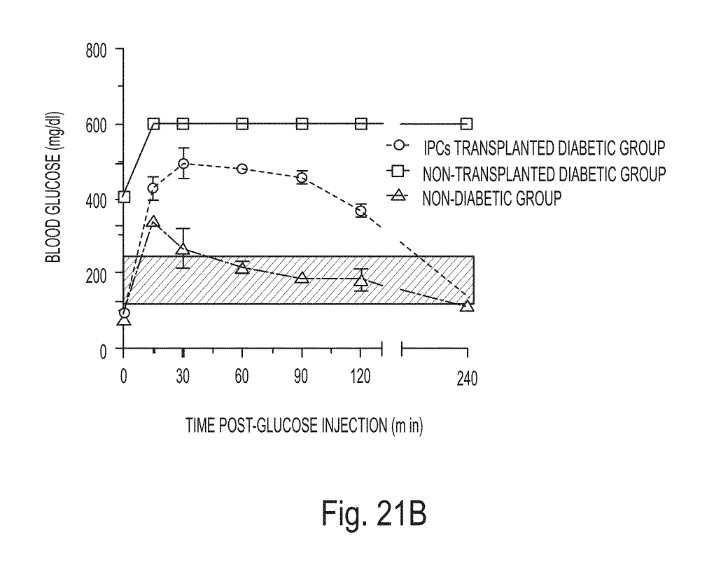

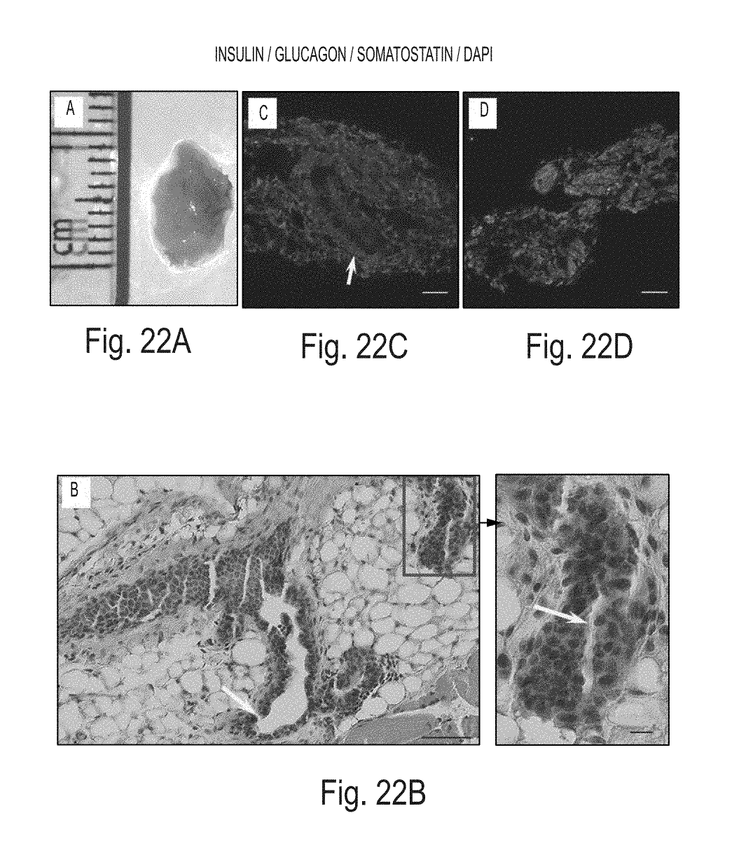

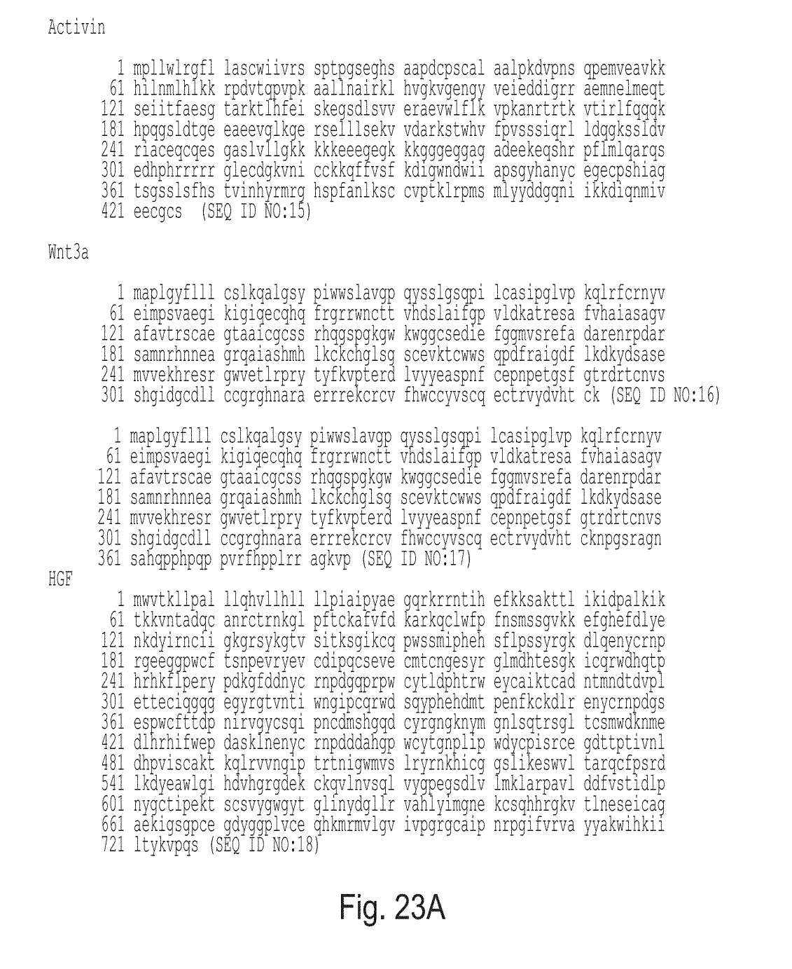

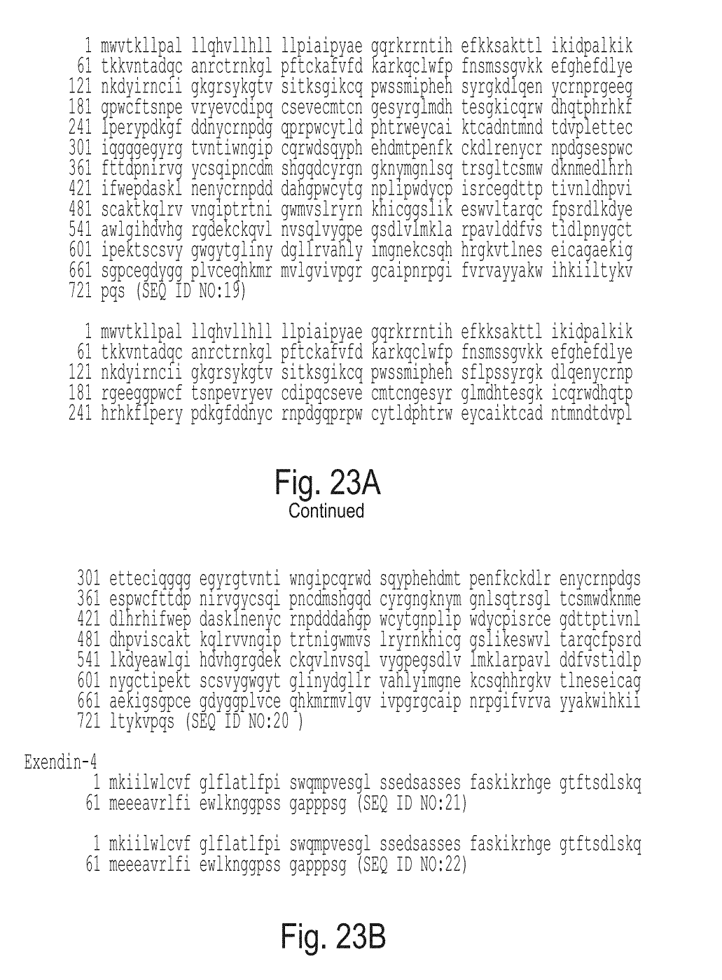

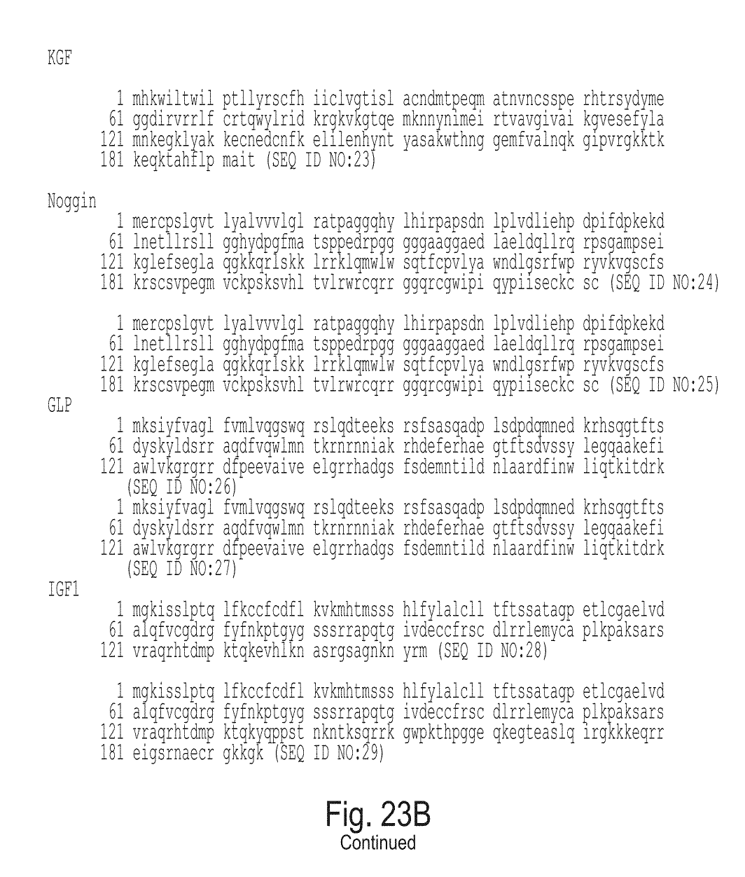

To determine whether the IPCs are functional in vivo, immunodeficient Rag2^{-/-}γ_{c}^{-/-} mice were made diabetic by multiple low doses of STZ. Mice were injected s.c. with 1.2-1.4×10⁶ IPCs in the shoulder region. Remarkably, the hyperglycemia plateaued in less than 2 weeks after transplantation and started to rapidly fall. Within 4 weeks, mice were either normoglycemic or achieved near normoglycemia. None of the 8 mice died or developed teratomas, FIG. 21A. All mice normalized blood glucose levels. Additionally, mice that showed stable correction of hyperglycemia were subjected to a glucose tolerance test (n=4), in which they received a supraphysiological glucose bolus i.p. Remarkably, in contrast to non-transplanted diabetic mice, which failed to correct hyperglycemia, IPC-transplanted mice completely recovered to normoglycemia in 4 hours (FIG. 20B). This is evidence for how the IPCs endowed these formerly diabetic mice with the ability to tolerate and manage glucose spikes. However, the correction of hyperglycemia was significantly delayed compared to nondiabetic control mice, which is also evident by computing the “Area Under the Curve” for the three treatment groups (FIG. 21C). This delayed correction of glucose levels could also be due to the fact that our transplant was still too small to correct a large bolus of glucose rapidly. Still, the complete return to normoglycemia in IPC-transplanted mice is striking evidence of the remarkable ability of these cells to manage supra-physiological glucose spikes. Excision of the transplanted cells after 8 weeks of s.c. transplantation revealed an organoid (FIG. 22A) that showed glandular morphology of cells surrounded by adipocytes and the presence of duct-like lumens (FIG. 22B). The morphology of these cells was highly similar to H&E staining shown of 10 week old organoids derived using kidney-capsule transplanted IPCs that were generated from human ES cells (Rezania et al., 2014). Interestingly, parts of the organoid were identified that stained positive for insulin only, (FIG. 22C), and other areas that expressed both insulin and somatostatin (FIG. 22D). Glucagon staining remained negative. Thus, T1D iPS cells differentiated through this protocol generate vascularized organoids that consist of insulin-expressing cells after transplantation into mice.

### Discussion

A robust protocol was established for the generation of IPCs from either healthy or T1D iPS cells. These findings are an enormous advance from prior protocols that generally yielded only 10-15% insulin⁺ cells from human ES cells or iPS cells derived from nondiabetic patients (Raikwar et al., 2015; D'Amour et al., 2006; Kroon et al., 2008; Rezania et al., 2012; Xie et al., 2013; Zhang et al., 2009; Pagliuca et al., 2014; Rezania et al., 2014). The present protocol is simple, highly reproducible and does not require exorbitant amounts of expensive reagents. Here, a system was used to generate a virtually pure population of CXCR4⁺ Sox17⁺ DE cells that did not express PDGFR-α, which marks mesodermal and mesendodermal cells (Tada et al., 2005). These cells were then driven through four more developmental stages in a 3D platform to yield >95% Pdx1⁺ cells and >50% insulin⁺ IPCs. These cells were organized in compact cell clusters that resemble islets and expressed insulin as determined by a glucose stimulation assay, flow cytometry, qRT-PCR and immunofluorescence. Although we detected some somatostatin expressing cells and insulin producing cells, glucagon was not detectable. The early differentiation of T1D and ND iPS cells into DE cells (Stage 1) was equivalent but downstream 3D differentiation of T1D iPS cells was impaired. After obtaining the gene expression data showing the impaired expression of Pdx1 in T1D differentiating cells, we reasoned that there were epigenetic barriers that hindered the expression of critical genes for pancreatic β-cell specification. Thus, it was hypothesized that using epigenetic modifiers such as 5-aza-DC would allow for the expression of Pdx1 and downstream genes, which altogether would result in a high yield of insulin⁺ IPCs from T1D iPS cells. 5-aza-DC was highly effective in improving the differentiation of T1D iPS cells into IPCs.

Confirmation that the cells are true β-cells comes from the transplantation data which show very rapid correction of hyperglycemia in diabetic mice. In comparison to all other data published by others, the cells abrogate the rise in hyperglycemia and rapidly induce normoglycemia in 4 weeks. In a clinical situation, this would be highly desirable.

The present findings are highly significant since iPS cell-based therapy for T1D will, in all likelihood, involve the patient's own somatic cell-derived iPS cells^{4,5}. With all prior reports using human ES cells or iPS cells derived from healthy subjects (Raikwar et al., 2015; D'Amour et al., 2006; Kroon et al., 2008; Rezania et al., 2012; Xie et al., 2013; Zhang et al., 2009; Pagliuca et al., 2014; Rezania et al., 2014), the present studies demand a better understanding of the influence of the disease state of the patient from which iPS cells are derived on the differentiation of these iPS cells into IPCs. Here, a highly efficient protocol for inducing directed derivation of IPCs from T1D patient-derived iPS cells was demonstrated.

### REFERENCES

Abi-Hanna et al., Transplantation, 45:610 (1988).
Alipio et al., Proc. Natl. Acad. Sci. USA, 107:13426 (2010).
Boheler et al., Circulation Res., 91:189 (2002).
Bonde et al., PLoS One, 3:e3212 (2008).
Brissova et al., J. Histochem. Cytochem., 53:1087 (2005).
Chen et al., Development, 140:675 (2013).
Chetty et al., Nat. Meth., 10:553 (2013).
Christman, Oncogene, 21:5483 (2002).
D'Addio et al., Diabetes, 63:3041 (2014).
D'Amour et al., Nat. Biotechnol., 24:1392 (2006).
Dao et al., Transplantation, _98 (10)_:_1040_(2014).
Drukker et al., Proc. Nat. Acad. Sci. USA, 99:9864 (2002).
Faendrich et al., Nat. Med., 8:171 (2002).
Fiorina et al., Endocr. Rev., 32:725 (2011).
Fiorina et al., J. Immunol., 183:993 (2009).
Firth et al., Proc. Nat. Acad. Sci. USA, 111:E1723 (2014).
Gellibert et al., J. Med. Chem., 47:4494 (2004).
Greggio et al., Development, 140:4452 (2013).
Groppe et al., Nature, 420:636 (2002).
Guo et al., Diabetes, 62:1581 (2013).
Hua et al., PLoS One, 9:e102198 (2014).
Hughes et al., Proteomics, 10:1886 (2010).
Kelly et al., Nat. Biotech., 29:750 (2011).
Kim et al., Blood, 121:5167 (2013).
Kleinman and Martin, Semin. Cancer Biol., 15:378 (2005).
Kroon et al., Nat. Biotech., 26:443 (2008).
Lancaster et al., Nature, 501:373 (2013).
Leroux et al., Can. J. Diabetes, 38:62 (2014).
Loh et al., Cell Stem Cell, 14:237 (2014).
Luo et al, Metabolism, 62:90 (2013).
Manzar et al., Immunol. Res., _59(1-3)_:_279_(2014).
Mfopou et al., Diabetes, 59:2094 (2010).
Miyoshi et al., Cell Stem Cell, 8:633 (2011).
Murtaugh, Development, 134:427 (2007).
Murtaugh et al., Proc. Nat. Acad. Sci. USA, 100:14920 (2003).
Nasr et al., Pharmacol. Res., _98_:_31_(2015).
Nishikawa et al., Nat. Rev. Mol. Cell Biol., 9:725 (2008).
Norris and Carr, in Vertebrate Endocrinology (Fifth Edition) (ed David O. NorrisJames A. Carr) pg. 443 (Academic Press, 2013).
Okita et al., Nat. Protocols, 5:418 (2010).
Otonkoski et al., J. Clin. Invest., 92:1459 (1993).
Pagliuca et al., Cell, 159:428 (2014).
Parnaud et al., Diabetes, 55:1413 (2006).
Pennarossa et al., Proc. Natl. Acad. Sci. USA, 110:8948 (2013).
Raikwar and Zavazava, Methods Mol. Biol., 590:317 (2009).
Raikwar and Zavazava, Transplantation Res., 1:19 (2012).
Raikwar and Zavazava, Transplantation, 91:11 (2011).
Raikwar et al., PLoS One, 10:e0116582 (2015).
Rezania et al., Diabetes, 61:2016 (2012).
Rezania et al., Nat. Biotechnol., 32:1121 (2014).
Robinton and Daley, Nature, 481:295 (2012).
Schaffer et al., PLoS Genetics, 9:e1003274 (2013).
Schulz et al., PLoS One, 7:e37004 (2012).
Shahjalal et al., J. Mol. Cell Biol., _6 (5)_:_394_(2014).
Shiroi et al., Stem Cells, 20:284 (2002).
Spence and Wells, Dev. Dyn., 236:3218 (2007).
Stoffers et al., Nat. Genet., 15:106 (1997).
Tada et al., Development, 132:4363 (2005).
Takahashi and Yamanaka, Cell, 126:663 (2006).
Takahashi et al., Cell, 131:861 (2007).
Takebe et al., Nature, 499:481 (2013).
Takeuchi et al., Sci. Rep., 4:4488 (2014).
Taylor et al., Cell Reports, 4:1262 (2013).
Thatava et al., Gene Ther., 18:283 (2011).
Withers et al., Nature Genetics, 23:32 (1999).
Xie et al., Cell Stem Cell, 12:224 (2013).
Ye et al., Diabetes, 55,742 (2006).
Yechoor and Chan, Mol. Endocr., 24:1501 (2010).
Zhang et al., Cell Res., 19:429 (2009).

All publications, patents and patent applications are incorporated herein by reference.

While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prepare insulin secreting cells from a skin sample from a mammal, comprising:
providing a sample of skin cells from a mammal; subjecting the skin cells to conditions that convert the skin cells to induced pluripotent stem cells; culturing the induced pluripotent stem cells stepwise under conditions that induce differentiation to definitive endodermal cells, wherein the steps include culturing the cells in a gelatinous protein mixture and optionally applying a demethylation agent; and
treating the definitive endodermal cells to a plurality of agents that are sequentially applied and which result in stepwise differentiation of the definitive endodermal cells to insulin secreting cells.

2. The method of claim 1 wherein the cells are human cells.

3. The method of claim 1 wherein the mammal is a human that has type 1 diabetes.

4. The method of claim 1 wherein the treating includes differentiating the definitive endodermal cells to posterior foregut cells, differentiating the posterior foregut cells to pancreatic endodermal or progenitor cells, differentiating the pancreatic endodermal or progenitor cells to endocrine precursors, and differentiating the endocrine precursor cells to insulin producing cells.

5. The method of claim 1 wherein the cells are treated with at least one of a KGF receptor (KGFR) agonist, L-ascorbic acid or an analog thereof or a Rho-associated kinase inhibitor, or any combination thereof.

6. The method of claim 5 wherein the cells are treated with at least one of keratinocyte growth factor (KGF), L-ascorbic acid, or Y27632, or any combination thereof.

7. The method of claim 1 wherein the cells are treated with at least one of a Smo inhibitor and Sonic Hedgehog signaling pathway antagonist, retinoic acid or an analog thereof, an inactivator of TGF-beta superfamily signaling proteins, B27, a PKC activator, L-ascorbic acid or an analog thereof, or a KGFR agonist, or any combination thereof.

8. The method of claim 7 wherein the cells are treated with at least one of SANT-1, retinoic acid, Noggin, B27, TPB, L-ascorbic acid, or keratinocyte growth factor, or any combination thereof.

9. The method of claim 1 wherein the cells are treated with at least one of an inhibitor of TGF-beta RI kinase, an inactivator of TGF-beta superfamily signaling proteins, B27 Supplement, GLP1 or an analog thereof, a Smo inhibitor and antagonist of Sonic Hedgehog signaling, retinoic acid or an analog thereof, an inhibitor of gamma-secretase complex, or heparin or an analog thereof, or any combination thereof, followed by treatment with an inhibitor of TGF-beta RI kinase, an inactivator of TGF-beta superfamily signaling proteins, B27 Supplement, GLP1 or an analog thereof, an inhibitor of gamma-secretase complex, or heparin or an analog thereof, or any combination thereof.

10. The method of claim 9 wherein the cells are treated with at least one of ALK5i, Noggin, B27 Supplement, glucagon like peptide-1 (GLP1), SANT1, retinoic acid, DAPT or heparin, or any combination thereof, followed by treatment with ALK5i, Noggin, B27 Supplement, GLP1, DAPT, heparin, or T3, or any combination thereof.

11. The method of claim 1 wherein the cells are treated with at least one of nicotinamide or an analog thereof, IGF-1 or an analog thereof, GLP-1 or an analog thereof, an inhibitor of TGF-beta RI kinase, T3 or an analog thereof, or heparin or an analog thereof, or any combination thereof.

12. The method of claim 11 wherein the cells are treated with at least one of nicotinamide, IGF-1, GLP-1, ALK5i, T3, or heparin, or any combination thereof.

13. The method of claim 1 wherein a demethylation agent is applied.

14. The method of claim 13 wherein the demethylation agent is applied during differentiation to definitive endodermal cells.

15. The method of claim 1 wherein the insulin secreting cells, once transplanted, are glucose sensitive with one to two weeks.

16. The method of claim 1 wherein the insulin secreting cells express insulin at levels that are at least 30% that of insulin secreting cells in a mammal that is not diabetic.

17. The method of claim 1 wherein the induced pluripotent stem cells are treated with at least one of Activin A or Wnt3a, or both, thereby providing definitive endodermal cells; wherein the definitive endodermal cells are treated with at least Activin A, introduced to a gelatin coated substrate and treated at least one of keratinocyte growth factor, Noggin, or B27 having insulin but lacking vitamin A, or any combination thereof, thereby providing pancreatic endoderm cells; and the pancreatic endoderm cells are treated with at least one of HGF, exendin-4, or nicotinamide, or any combination thereof.

18. The method of claim 1 wherein the induced pluripotent stem cells are cultured with at least one of a TGF-beta family member or a Wnt family member, or both, thereby providing definitive endodermal cells.

19. The method of claim 1 wherein the definitive endodermal cells are introduced to a gelatin coated substrate and treated with at least one of a keratinocyte growth factor receptor agonist, an inactivator of TGF-beta superfamily member signaling proteins, or B27 having insulin but lacking vitamin A, or any combination thereof, thereby providing pancreatic endoderm cells; wherein the pancreatic endoderm cells are treated with at least one of HGF or an analog thereof, exendin-4 or an analog thereof, or nicotinamide or an analog thereof, or any combination thereof, thereby providing pancreatic endocrine precursors; and wherein the pancreatic endocrine precursors are treated with an inactivator of TGF-beta superfamily member signaling proteins.

20. The method of claim 1 wherein the definitive endodermal cells are treated with at least a TGF-beta family member and introduced to a gelatin coated substrate and are treated at least one of a keratinocyte growth factor receptor agonist, an inactivator of TGF-beta superfamily member signaling proteins, or B27 having insulin but lacking vitamin A, or any combination thereof, thereby providing pancreatic endoderm cells; and treating the pancreatic endoderm cells with at least one of HGF or an analog thereof, exendin-4 or an analog thereof, or nicotinamide or an analog thereof, or any combination thereof.
